(19) United States
(12) Patent Application Publication
(10) Pub. No.: US 2022/0369963 A1
(43) Pub. Date: Nov. 24, 2022

(54) BIOLOGICAL INFORMATION MEASURING DEVICE AND BIOLOGICAL INFORMATION MEASURING METHOD

(71) Applicant: RICOH COMPANY, LTD., Tokyo (JP)

(72) Inventors: Yoshihiro OBA, Miyagi (JP); Ryosuke KASAHARA, Kanagawa (JP); Yoshio WADA, Miyagi (JP); Toshihide SASAKI, Miyagi (JP)

(21) Appl. No.: 17/771,491

(22) PCT Filed: Oct. 26, 2020
(86) PCT No.: PCT/JP2020/040128

§ 371 Date: Apr. 25, 2022

(30) Foreign Application Priority Data
Oct. 28, 2019 (JP) 2019-195631
Oct. 28, 2019 (JP) 2019-195632
Oct. 28, 2019 (JP) 2019-195634
Oct. 28, 2019 (JP) 2019-195635
Nov. 6, 2019 (JP) 2019-201352
Sep. 28, 2020 (JP) 2020-162222

(51) Int. Cl. A61B 5/145; A61B 5/1455

(52) U.S. Cl. CPC A61B 5/14532; A61B 5/1455

(57) ABSTRACT

A biological information measuring device (100a) includes a light source configured to emit probe light; a total reflection member (16) configured to totally reflect the probe light with the total reflection member (16) brought into contact with a subject (S) to be measured; a light intensity detector configured to detect light intensity of the probe light reflected from the total reflection member (16); a biological information output unit (2a) configured to output biological information, the biological information being acquired based on the light intensity; and a display unit (506) configured to display the light intensity or an absorbance of the probe light, the absorbance being acquired based on the light intensity. Preferably a pressure detector is provided configured to detect a pressure of the subject (S) with respect to the total reflection member (16).

### TECHNICAL FIELD

The disclosure herein relates to a biological information measuring device and a biological information measuring method.

### BACKGROUND ART

The number of patients with diabetes has increased worldwide, and noninvasive blood glucose level measuring techniques without blood sampling are desired. A variety of methods have been proposed for measuring biological information such as blood glucose levels using light, such as methods using near-infrared light, mid-infrared light, and Raman spectroscopy. Of these, the methods using mid-infrared light can increase the measurement sensitivity higher than the methods using the near-infrared light. This is because the mid-infrared region is the fingerprint region where glucose absorption is high.

As light sources for the mid-infrared region, light emitting devices such as Quantum Cascade Lasers (QCL) are available. However, such light sources often require the number of laser light sources corresponding to the number of wavelengths used. In view of downsizing of devices, it is desirable to reduce the wavelengths of the mid-infrared region to a few wavelengths.

In order to accurately measure glucose concentration in a specific wavelength region such as the mid-infrared region by using an ATR (Attenuated Total Reflection) method, Patent Document 1, for example, proposes a method for using the wavelengths of the absorption peaks of glucose (1035 cm⁻¹, 1080 cm⁻¹, and 1110 Cm⁻¹).

According to such a method by using ATR, the measurement is performed by bringing a total reflection member, such as an ATR prism, into contact with a subject to be measured. However, in this method, biological information, such as glucose concentration, may fail to be measured accurately due to fluctuation in a contact state between the subject and the total reflection member.

In order to overcome this, Patent Document 2, for example, discloses a technique for adjusting a contact area between a total reflection member such as an ATR prism, and a subject to be measured, upon bringing the total reflection member into contact with the subject to be measured.

Further, Patent Document 3, for example, discloses a technique using a pressure sensor to detect a contact pressure applied to a subject to be measured with the subject being in contact with a total reflection member so as to acquire biological information in response to the contact pressure being within a predetermined range.

### CITATION LIST

### Patent Literature

[PTL 1] Japanese Patent No. 5376439
[PTL 2] Japanese Unexamined Patent Application Publication No. H11-188009
[PTL 3] Japanese Unexamined Patent Application Publication No. 2015-173935

### SUMMARY OF INVENTION

### Technical Problem

However, the related art techniques may fail to accurately measure biological information because those techniques can adjust a contact area or a contact pressure between the total reflection member and the subject to be measured.

It is an object of the present invention to accurately measure biological information.

### Solution to Problem

According to an aspect of an embodiment, a biological information measuring device includes a light source configured to emit probe light; a total reflection member configured to totally reflect the probe light with the total reflection member brought into contact with a subject to be measured; a light intensity detector configured to detect light intensity of the probe light reflected from the total reflection member; a biological information output unit configured to output biological information, the biological information being acquired based on the light intensity; and a display unit configured to display the light intensity or an absorbance of the probe light, the absorbance being acquired based on the light intensity.

### Advantageous Effect of the Invention

According to at least one aspect of embodiments of the present invention, biological information can be accurately measured.

### BRIEF DESCRIPTION OF DRAWINGS

FIG. 1 is a diagram illustrating an overall configuration of a blood glucose level measuring device according to an embodiment.

FIG. 2 is a diagram illustrating an action of an Attenuated Total Reflection (ATR) prism.

FIG. 3 is a perspective diagram illustrating a structure of the ATR prism.

FIG. 4 is a perspective diagram illustrating a structure of a hollow fiber.

FIG. 5 is a block diagram illustrating a hardware configuration example of a processor according to the embodiment.

FIG. 6 is a block diagram illustrating a functional configuration example of a processor according to an embodiment.

FIG. 7A is a diagram illustrating an example of a switching operation when first probe light is used.

FIG. 7B is a diagram illustrating an example of a switching operation when second probe light is used.

FIG. 7C is a diagram illustrating an example of a switching operation when third probe light is used.

FIG. 8 is a flowchart illustrating an example of an operation of a blood glucose level measuring device according to an embodiment.

FIG. 9A is a graph illustrating probe light intensities of a comparative example.

FIG. 9B is a graph illustrating probe light intensities each varying in three or more steps.

FIG. 10A is a graph illustrating a cross-sectional light intensity distribution of probe light.

FIG. 10B is a graph illustrating a cross-sectional light intensity distribution of FIG. 10A of probe light after positional shift.

FIG. 10C is a graph illustrating a cross-sectional light intensity distribution of probe light including speckles.

FIG. 10D is a graph illustrating a cross-sectional light intensity distribution of FIG. 10C after positional shift.

FIG. 11A is a diagram illustrating total reflection of probe light when an incident surface is a flat surface.

FIG. 11B is a diagram illustrating total reflection of probe light when the incident surface is a diffusion surface,

FIG. 11C is a diagram illustrating total reflection of probe light when the incident surface is a diffusion surface.

FIG. 11D is a diagram illustrating total reflection of probe light when the incident surface is a concave diffusion surface.

FIG. 11E is a diagram illustrating total reflection of probe light when the incident surface is a convex surface.

FIG. 12A is a diagram illustrating relative positional shifts of the first and second hollow optical fibers with respect to the ATR prism when the ATR prism is not in contact with the living body.

FIG. 12B is a diagram illustrating relative positional shifts of the first and second hollow optical fibers with respect to the ATR prism when a first total reflection surface of the ATR prism is in contact with the living body.

FIG. 12C is a diagram illustrating relative positional shifts of the first and second hollow optical fibers with respect to the ATR prism when a second total reflection surface of the ATR prism is in contact with the living body.

FIG. 13 is a diagram illustrating first and second hollow optical fibers and a support member of the ATR prism.

FIG. 14A is a graph illustrating an example of light source drive current according to a comparative example.

FIG. 14B is a graph illustrating an example of high-frequency modulated light source drive current.

FIG. 15A is a diagram illustrating a configuration example of a measuring unit in a blood glucose level measuring device according to a first embodiment.

FIG. 15B is a diagram illustrating an arrangement of the measuring unit, a camera, and a display in the blood glucose level measuring device according to the first embodiment.

FIG. 16A is a diagram illustrating a configuration example in which one pressure sensor is disposed.

FIG. 16B a diagram illustrating a configuration example in which two pressure sensors are disposed at opposite ends of an ATR prism.

FIG. 16C a diagram illustrating a configuration example in which a plurality of pressure sensors is disposed.

FIG. 17A is a diagram illustrating an arrangement of an ATR prism with respect to the lip of a living body before the ATR prism is in contact with the lip.

FIG. 17B is a diagram illustrating an arrangement of an ATR prism with respect to the lip of a living body when the living body holds the ATR prism in his mouth.

FIG. 18 is a block diagram illustrating a functional configuration example of a processor according to a first embodiment.

FIG. 19 is a diagram illustrating an example of a display screen that displays light intensity and absorbance.

FIG. 20 is a diagram illustrating an example of a display screen that displays a contact pressure and a contact region.

FIG. 21A is a flowchart illustrating a part of a process performed by a processor according to the first embodiment.

FIG. 21B is the flowchart illustrating another part of the process performed by the processor according to the first embodiment.

FIG. 22 is a variation of a flowchart illustrating a process performed by the processor according to the first embodiment.

### DESCRIPTION OF EMBODIMENTS

Hereinafter, embodiments will be described with reference to the accompanying drawings. In each drawing, the same parts of the same components are indicated by the same reference numerals, and duplicated descriptions will be omitted.

### Description of the Terms of Embodiments

(Mid-Infrared Region)

The mid-infrared region refers to a wavelength region of 2 to 14 μm. The mid-infrared region is an example of a specific wavelength region.

(Probe Light)

Probe light refers to light that is used for measuring absorbance and biological information. According to an embodiment, probe light corresponds to light that is completely reflected by a total reflection member, is attenuated by a living body, and is then detected by a light intensity detector.

(ATR Method)

The ATR method (attenuated total reflection method or total reflection absorption method) is a method for acquiring absorption spectrum of a subject to be measured by using a field penetration from a total reflection surface when total reflection occurs in the total reflection member that is in contact with the subject to be measured. An ATM prism is an example of the total reflection member.

(Absorbance)

Absorbance is a dimensionless amount that indicates degrees of reduction in light intensity when light passes through a subject. According to an embodiment, attenuation by a living body in the field penetration from the total reflection surface is measured as absorbance by the ATR (Attenuated Total Reflection) method.

(Blood Glucose Level)

A blood glucose level refers to the concentration of glucose (glucose) in the blood.

(Detection Value)

According to an embodiment, a detection value is defined as a value detected by the light intensity detector.

(Wavenumber)

The relationship between wavelength λ (μm) and wavenumber k (cm⁻¹) is represented by k=10000/λ.

Hereinafter, an embodiment will be described with reference to an example of a blood glucose level measuring device (an example of a biological information measuring device) for measuring a blood glucose level (an example of biological information) based on absorbance, which is measured using an ATR prism (an example of a total reflection member).

### Embodiment

First, a blood glucose level measuring device 100 according to an embodiment will be described.

According to an embodiment, probe light beams having different wavelengths in the mid-infrared region is injected into a total reflection member disposed in contact with a living body, absorbance of each of the probe light beams is acquired based on the ATR method, and the blood glucose level is measured based on the acquired absorbance.

<Overall Configuration Example of Blood Glucose Level Measuring Device 100>

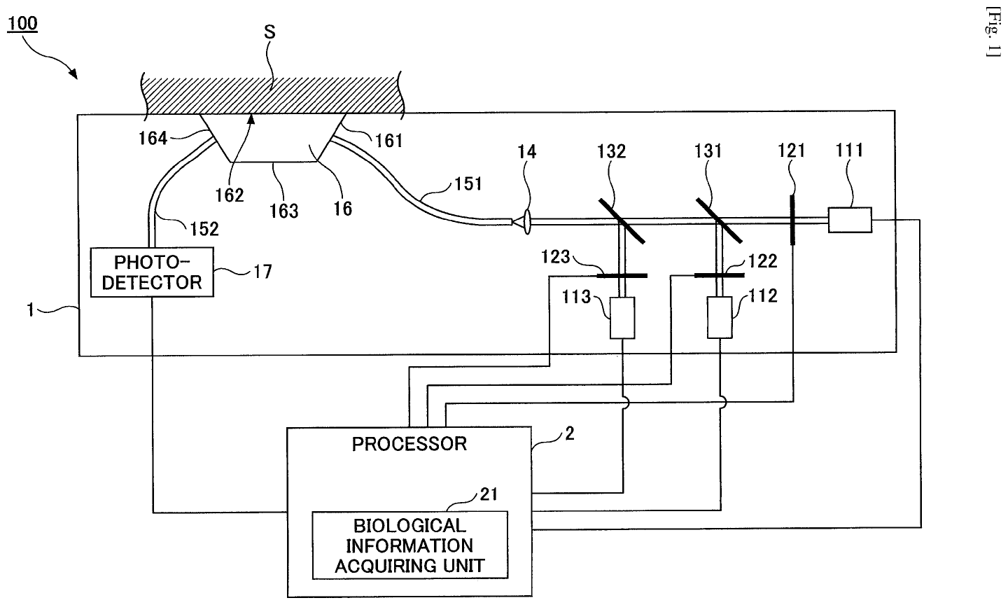

FIG. 1 is a diagram illustrating an overall configuration example of the blood glucose level measuring device 100. As illustrated in FIG. 1, the blood glucose level measuring device 100 includes a measuring unit 1 and a processor 2.

The measuring unit 1 is an optical head for performing an ATR method. The measuring unit 1 is configured to output a detection signal of biologically attenuated probe light to the processor 2. The processor 2 is a processing device configured to acquire absorbance data based on the detection signal, and also to acquire a blood glucose level based on the absorbance data and output the acquired blood glucose level.

The measuring unit 1 includes a first light source 111, a second light source 112, a third light source 113, a first shutter 121, a second shutter 122, and a third shutter 123. The measuring unit 1 further includes a first half mirror 131, a second half mirror 132, a coupling lens 14, a first hollow optical fiber 151, an ATR prism 16, a second hollow optical fiber 152, and a photodetector 17.

The processor 2 includes a biological information acquisition unit 21. The first light source 111, the second light source 112, and the third light source 113 in the measuring unit 1 are respective quantum cascade lasers that are electrically coupled to the processor 2. The first light source 111, the second light source 112, and the third light source 113 in the measuring unit 1 are also configured to emit laser light in the mid-infrared region, in response to a control signal from the processor 2.

According to the embodiment, the first light source 111 emits laser light having a wavenumber of 1050 cm⁻¹ as first probe light, the second light source 112 emits laser light having a wavenumber of 1070 cm⁻¹ as second probe light, and the third light source 113 emits laser light having a wavenumber of 1100 cm⁻¹ as third probe light.

Laser light with wavenumbers of 1050 cm⁻¹, 1070 cm⁻¹, and 1100 cm⁻¹ correspond to the wavenumbers of respective glucose absorption peaks, and the absorbance can be measured using these wavenumbers to accurately measure glucose concentration based on the absorbance.

The first shutter 121, the second shutter 122, and the third shutter 123 are respective electromagnetic shutters electrically coupled to the processor 2. The first shutter 121, the second shutter 122, and the third shutter 123 are controlled to open or close in response to a control signal from the processor 2.

When the first shutter 121 is opened, the first probe light from the first light source 111 passes through the first shutter 121 to reach the first half mirror 131. When the first shutter 121 is closed, the first probe light is shielded by the first shutter 121, and thus does not reach the first half mirror 131.

When the second shutter 122 is opened, the second probe light from the second light source 112 passes through the second shutter 122 to reach the first half mirror 131. When the second shutter 122 is closed, the second probe light is shielded by the second shutter 122, and thus does not reach the first half mirror 131.

Similarly, when the third shutter 123 is opened, the third probe light from the third light source 113 passes through the third shutter 123 to reach the second half mirror 132. When the third shutter 123 is closed, the third probe light is shielded by the third shutter 123, and thus does not reach the second half mirror 132.

The first half mirror 131 and the second half mirror 132 are optical elements configured to transmit a portion of incident light, and to reflect a remaining portion of the incident light. Such optical elements may be configured by disposing an optical thin film on an optically transparent substrate so as to transmit a portion of the incident light, and reflect the remaining portion of the incident light.

However, the optical elements are not necessarily limited to an optical thin film, but may be configured by forming a diffractive structure to transmit a portion of incident light through an optically transparent substrate, and to reflect (diffract) the remaining portion of the incident light. The use of diffractive structure is suitable because the diffractive structure will prevent optical absorption.

The first half mirror 131 transmits the first probe light that passes through the first shutter 121, and reflects the second probe light that has passed through the second shutter 122. The second half mirror 132 also transmits each of the first probe light and the second probe light, and reflects the third probe light that has passed through the third shutter 123.

It is preferable that the light intensity ratio of the transmitted light to the reflected light in each of the first and second half mirrors 131 and 132 be approximately 1:1; however, the light intensity ratio can be adjusted according to the probe light intensity emitted by each light source or the like.

The first to third probe light transmitted through the first half mirror 131 or the second half-mirror 132 is guided into the first hollow optical fiber 151 via a coupling lens 14, and is then propagated into the first hollow optical fiber 151 to be optically guided into the ATR prism 16 via an incident surface 161 of the ATR prism 16.

The ATR prism 16 is an optical prism that propagates first to third probe light toward an emission surface 164 while totally reflecting the first to third probe light incident from the incident surface 161, and that emits the first to third probe light from the emission surface 164. As illustrated in FIG. 1, the ATR prism 16 is disposed on the first total reflection surface 162 that is in contact with a living body S (an example of a subject to be measured).

The first to third probe light guided in the ATR prism 16 repeats total reflection by the first total reflection surface 162 and by the second total reflection surface 163 opposite to the first total reflection surface 162, and the first to third probe light is then guided into the second hollow optical fiber 152 via the emission surface 164.

The first to third probe light guided by the second hollow optical fiber 152 reaches the photodetector 17. The photodetector 17 is a detector configured to detect light having wavelengths in the mid-infrared region, and photoelectrically convert the received first to third probe light, and output to the processor 2 an electrical signal corresponding to the light intensity as a detection signal. The photodetector 17 includes a PD (Photo Diode) for infrared light, an MCT (Mercury Cadmium Telluride) sensing element, a bolometer, or the like. The photodetector 17 is an example of a light intensity detector. Hereinafter, when the first to third probe light is not distinguished, the probe light may be simply referred to as probe light.

The processor 2 is composed of an information processing device such as a PC (Personal Computer). The biological information acquisition unit 21 in the processor 2 acquires absorbance data of each probe light based on a detection signal acquired by the photodetector 17, acquires blood glucose level data of the living body based on the absorbance data, and outputs the blood glucose level data to a display device, a storage device, an external server, or the like.

Note that in order to clarify a configuration of the measuring unit 1, the measuring unit 1 is enclosed by a solid line in FIG. 1. However, the solid line does not illustrate a housing of the measuring unit 1. The ATR prism 16 is not housed within a housing, such that at least one of the first total reflection surface 162 or the second total reflection surface 163 of the ATR prism 16 is configured to come into contact with any portion of the living body.

<Action/Configuration of ATR Prism 16>

Next, an action of the ATR prism 16 will be described with reference to FIG. 2. As illustrated in FIG. 2, the ATR prism 16 in the measuring unit 1 is disposed in contact with a living body S. The first to the third probe light incident on the ATR prism 16 each undergo attenuation with respect to a corresponding infrared absorption spectrum of the living body S. The attenuated probe light is received by the photodetector 17. The photodetector 17 detects light intensity of each probe light. A detection signal is input to the processor 2, and the processor 2 acquires and outputs absorbance data and blood glucose level data, based on the detection signal.

The attenuated total reflection (ATR) with infrared spectroscopy (hereinafter called “infrared ATR method”) is useful for performing spectroscopic detection in the mid-infrared region, where glucose absorption intensity is obtained. The infrared ATR method utilizes a “field penetration”. The field penetration appears upon injection of probe light, i.e., infrared light, into the ATR prism 16 with a high refractive index, and total reflection occurs at an interface between the ATR prism 16 and an external environment (e.g., the living body S). When the measurement is performed while the ATR prism 16 is in contact with a living body S acting as a subject to be measured, the field penetration is absorbed by the living body S.

When infrared light with a wide wavelength range from 2 to 12 μcm is used as probe light, light having a wavelength derived from molecular vibrational energy of the living body S is absorbed, and optical absorption appears as a dip at the corresponding wavelength of the probe light transmitted through the ATR prism 16. This technique is particularly advantageous for infrared spectroscopy using weak power probe light because the technique allows a large amount of detected light to pass through the ATR prism 16.

When infrared light is used, the penetration depth of light that penetrates the living body S from the ATR prism 16 is only a few microns. Thus, the light does not reach the capillaries at a depth of several hundred microns. However, it is known that the blood plasma and other components leak into the skin and mucous cells as tissue fluid (interstitial fluid). The blood glucose level can be measured by detecting the glucose component present in the tissue fluid.

It is conceivable that the concentration of the glucose component in the tissue fluid increases at a position closer to the capillaries, such that the ATR prism 16 may need to be constantly pressed at a constant pressure during the measurement. Advantageously, according to an embodiment, a multiple reflection ATR prism with trapezoidal cross-sections are employed.

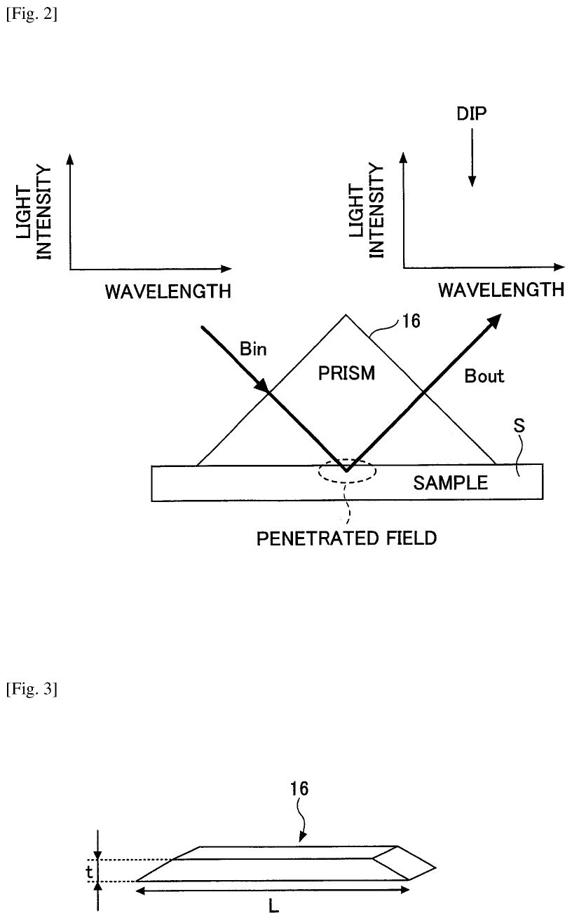

FIG. 3 is a perspective diagram illustrating the structure of the ATR prism according to an embodiment. As illustrated in FIG. 3, the ATR prism 16 is a trapezoidal prism. The greater the number of multiple reflections in the ATR prism 16, the more sensitive the detection of glucose. In addition, since a contact area of the ATR prism 16 with the living body S is large, detection value fluctuation due to a change in the pressure applied from the living body S to the ATR prism 16 can be minimized. The length L of the bottom of the ATR prism 16 is, for example, 24 mm. The thickness t is, for example, 1.6 mm or 2.4 mm being a value that enables multiple reflections.

As candidates for a material used for the ATR prism 16, a material that is not toxic to a human body and exhibits a high transmission characteristic at a wavelength of approximately 10 μcm being a glucose absorption band, may be given. Among the materials meeting these conditions, a ZnS (zinc sulfide) prism with a refractive index of 2.2 may be used. Such a ZnS prism exhibits a large optical penetration and is capable of detecting a greater depth. Unlike ZnSe (zinc selenide), which is commonly used as an infrared material, ZnS has been illustrated to be non-carcinogenic and is also used as a non-toxic dye (lithopone) in dental materials.

Of a typical ATR measuring device, the ATR prism is fixed to a relatively large device, such that a body part, which is a subject to be measured, is limited to a surface of the body such as the fingertips and forearms. However, the skin of these body parts is covered with a stratum corneum having a thickness of approximately 20 μcm, such that the detected glucose concentration may be small. In addition, the stratum corneum is affected by the secretion of sweat and sebum, both of which restrict the reproducibility of measurements. To overcome this restriction, the blood glucose level measuring device 100 employs a first hollow optical fiber 151 and a second hollow optical fiber 152 capable of transmitting probe light that is infrared light at low loss, where one end of each of the first and second hollow optical fibers 151 and 152 is in contact with the ATR prism 16.

One end of the first hollow optical fiber 151 is optically coupled to the incident surface 161 of the ATR prism 1, such that the emission light from the first hollow optical fiber 151 enters the incident surface 161 of the ATR prism 16.

One end of the second hollow optical fiber 152 is optically coupled to the emission surface 164 of the ATR prism 16 such that the emission light from the emission surface 164 of the ATR prism 16 is guided into the second hollow optical fiber 152.

The use of the ATR prism 16 enables the glucose measurement with the oral mucosa without a stratum corneum, or with the earlobes that are located relatively close to the skin surface, and are less affected by sweat or sebum.

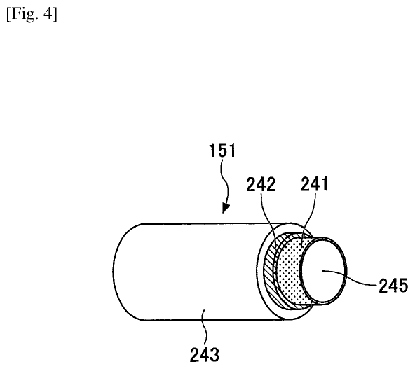

FIG. 4 is a perspective diagram illustrating an example of the structure of a hollow optical fiber used in the blood glucose level measuring device 100. Mid-infrared light having a relatively long wavelength for measuring glucose is absorbed by the glass in quartz glass optical fibers and cannot be transmitted. Various types of optical fibers for infrared transmission using special materials have been developed; however, these optical fibers are not used in the medical field due to toxicity, hygroscopicity, and chemical durability of the materials.

By contrast, of the first hollow optical fiber 151 and the second hollow optical fiber 152, a metallic thin film 242 and a dielectric thin film 241 are disposed in this order on the inner surface of the tube 243 being formed by a non-harmful material such as glass, plastic, and the like. The metallic thin film 242 is made of a less toxic material such as silver. The metallic thin film 242 is coated with a dielectric thin film 241 so as to exhibit chemical and mechanical durability. In addition, since a core 245 is air that does not absorb mid-infrared light, low-loss transmission of mid-infrared light can be achieved over a wide wavelength range.

<Configuration of Processor 2>

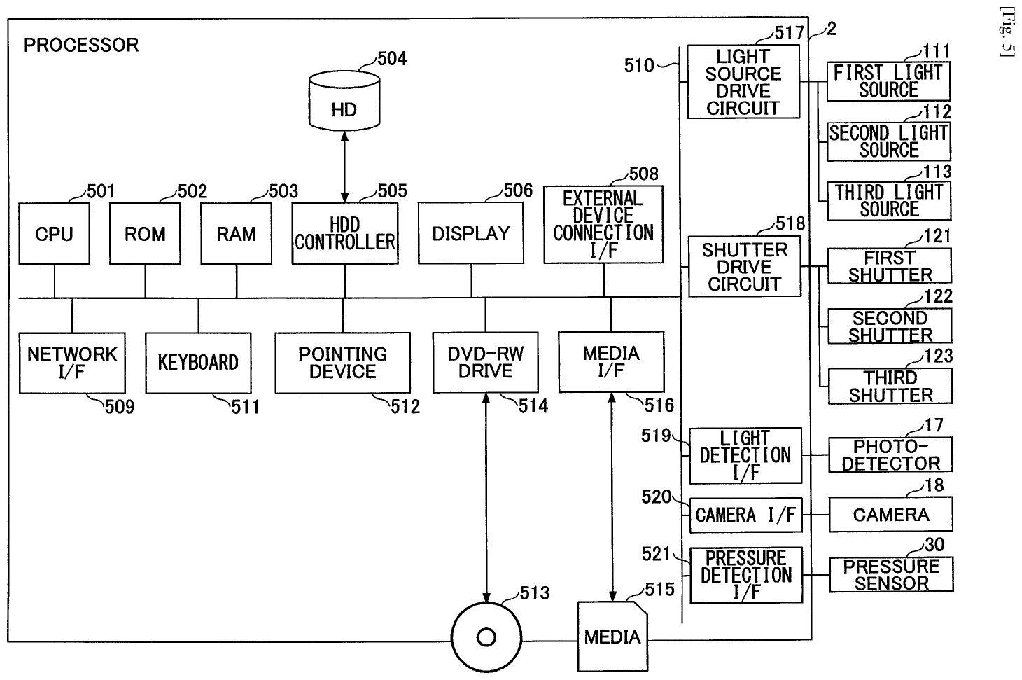

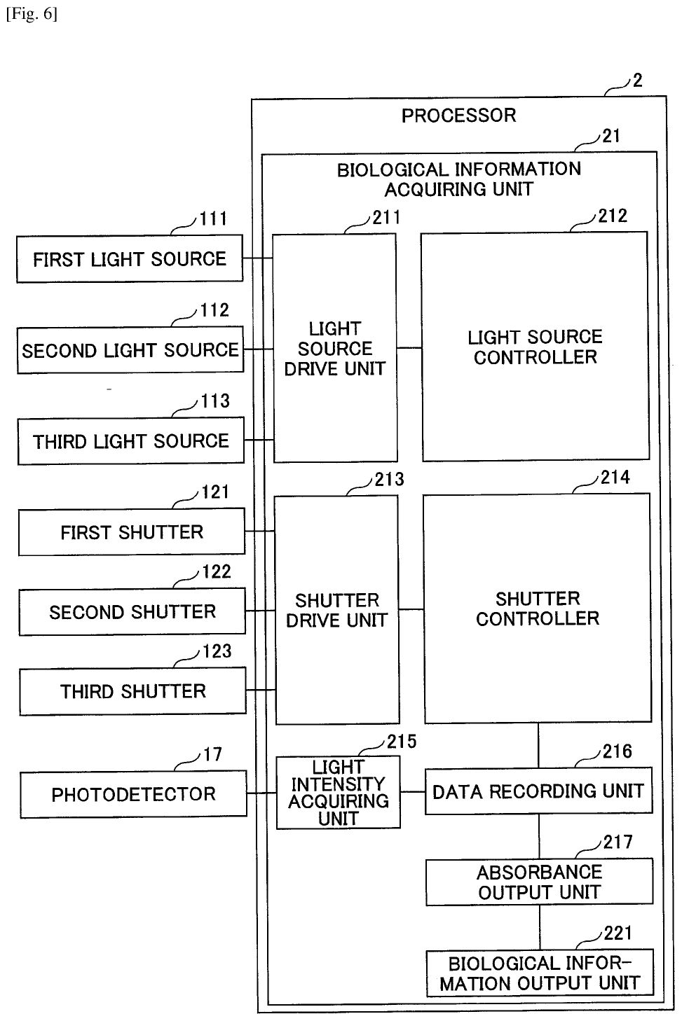

Next, a configuration of the processor 2 will be described with reference to FIGS. 5 and 6.

FIG. 5 is a block diagram illustrating an example of a hardware configuration of a processor 2 according to the embodiment. As illustrated in FIG. 5, the processor 2 includes a CPU (Central Processing Unit) 501, a ROM (Read Only Memory) 502, a RAM (Random Access Memory) 503, a HD (Hard Disk) 504, an HDD (Hard Disk Drive) controller 505, and a display 506. The processor 2 also includes an external device connection I/F (Interface) 508, a network I/F 509, a bus line 510, a keyboard 511, a pointing device 512, a DVD-RW (Digital Versatile Disk Rewritable) drive 514, a media I/F 516, a light source drive circuit 517, a shutter drive circuit 518, a photodetector I/F 519, a camera I/F 520, and a pressure detection I/F 521.

Of these, the CPU 501 controls operations of the entire processor 2. The ROM 502 stores a program used to drive the CPU 501, such as IPL (Initial Program Loader). The RAM 503 is used as a work area of the CPU 501.

The HD 504 stores various types of data such as a program. The HDD controller 505 controls reading or writing of various types of data with respect to the HD 504 according to the control of the CPU 501. The display 506 displays various types of information such as a cursor, menus, windows, characters, or images.

The external device connection I/F 508 is an interface for connecting various external devices. In this case, the external devices may be, for example, a USB (Universal Serial Bus) memory or a printer. The network I/F 509 is an interface for performing data communication using a communication network. The bus line 510 is an address bus, a data bus, or the like for electrically connecting components such as the CPU 501 illustrated in FIG. 5.

The keyboard 511 is also a type of an input unit with a plurality of keys for input of characters, numbers, various instructions, and the like. The pointing device 512 is a type of an input unit for selecting and executing various instructions, selecting a processing target, moving a cursor, and the like. The DVD-RW drive 514 controls reading or writing of various types of data with respect to the DVD-RW 513 acting as an example of a removable recording medium. The removable recording medium is not limited to the DVD-RW, but may be DVD-R, etc. The media I/F 516 controls reading or writing (storing) of data with respect to a recording medium 515, such as a flash memory.

The light source drive circuit 517 is an electrical circuit that is electrically coupled to each of the first light source 111, the second light source 112, and the third light source 113. The light source drive circuit 517 outputs a drive voltage for driving the first light source 111, the second light source 112, and the third light source 113 to emit infrared light in response to a control signal. The shutter drive circuit 518 is an electrical circuit that is electrically coupled to each of the first shutter 121, the second shutter 122, and the third shutter 123. The shutter drive circuit 518 outputs a drive voltage for driving the first shutter 121, the second shutter 122, and the third shutter 123 to open and close in response to a control signal.

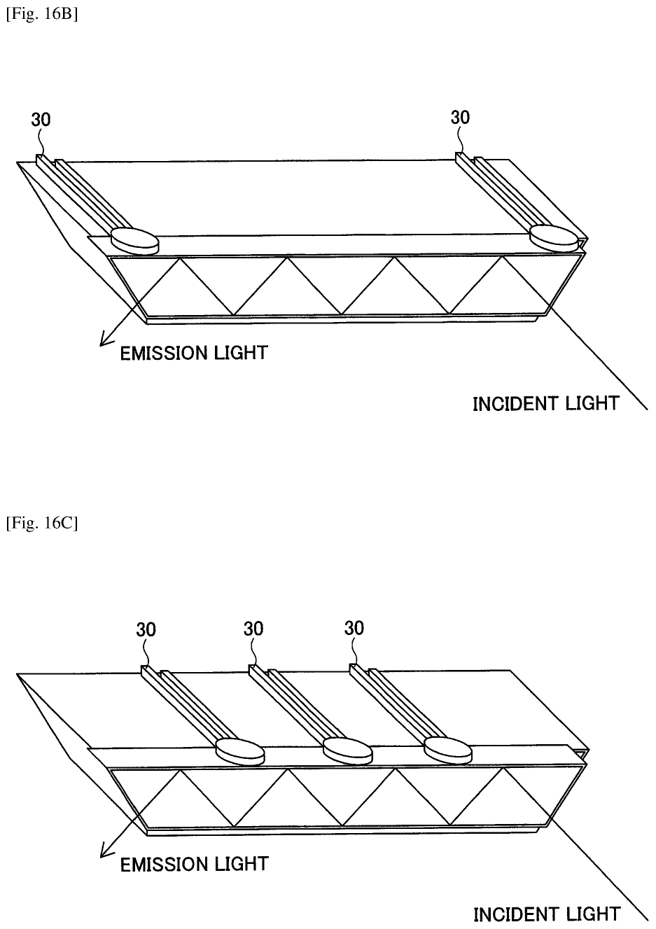

The photodetector I/F 519 is an electrical circuit such as an A/D (Analog/Digital) conversion circuit that functions as an interface for acquiring a detection signal of the photodetector 17. The camera I/F 520 is an electrical circuit that functions as an interface for acquiring images captured by the camera 18. The pressure detection I/F 521 is an electrical circuit such as an A/D conversion circuit that functions as an interface for acquiring a detection signal from the pressure sensor 30. The camera 18 and the pressure sensor 30 will be described later with reference to FIGS. 16 to 18.

Next, FIG. 6 is a block diagram illustrating a functional configuration example of a processor 2 according to an embodiment. As illustrated in FIG. 6, the processor 2 includes a biological information acquisition unit 21.

The biological information acquisition unit 21 includes a light source drive unit 211, a light source controller 212, a shutter drive unit 213, a shutter controller 214, a light intensity acquisition unit 215, a data recorder 216, an absorbance output unit 217, and a biological information output unit 221.

Of these, the function of the light source drive unit 211 is implemented by the light source drive circuit 517 or the like, the function of the shutter drive unit 213 is implemented by the shutter drive circuit 518 or the like, the function of the light intensity acquisition unit 215 is implemented by the light detection I/F 519 or the like, and the function of the data recorder 216 is implemented by the HD 504 or the like. The functions of the light source controller 212, the shutter controller 214, the absorbance output unit 217, and the biological information output unit 221 are implemented by executing a predetermined program by the CPU 501 or the like.

The light source drive unit 211 outputs a drive voltage based on a control signal input from the light source controller 212 so as to cause each of the first light source 111, the second light source 112, and the third light source 113 to emit infrared light. The light source controller 212 controls emission timing and intensity of the infrared light according to the control signal.

The shutter drive unit 213 outputs a drive voltage based on a control signal input from the shutter controller 214 to open or close the first shutter 121, the second shutter 122, and the third shutter 123. The shutter controller 214 controls the timing and duration of opening the shutters according to the control signal.

The light intensity acquisition unit 215 outputs to the data recorder 216 a detection value of the light intensity acquired by sampling a detection signal being continuously output by the photodetector 17 in a predetermined period. The data recorder 216 records a detection value input from the light intensity acquisition unit 215.

The absorbance output unit 217 acquires absorbance data by performing a predetermined calculation process based on the detection value read from the data recorder 216, and outputs the acquired absorbance data to the biological information output unit 221.

However, the absorbance output unit 217 may output the acquired absorbance data to an external device such as a PC through the external device connection I/F 508, or may output the acquired absorbance data to an external server through the network I/F 509 and the network. Alternatively, the absorbance output unit 217 may output the acquired absorbance data to the display 506 (see FIG. 5) for displaying the acquired absorbance data.

The biological information output unit 221 acquires blood glucose level data by performing a predetermined calculation process based on the absorbance data input from the absorbance output unit 217, and outputs the acquired blood glucose level data to a display 506 for displaying the blood glucose level data.

However, the biological information output unit 221 may output blood glucose level data to an external device such as a PC through the external device connection I/F 508, or the biological information output unit 221 may output blood glucose level data to an external server through the network I/F 509 and the network. The biological information output unit 221 may be configured to output the reliability of the blood glucose level measurement.

Since the technique disclosed in Japanese Unexamined Patent Application Publication No. 2019-037752, etc. can be applied to the process of acquiring blood glucose level data from absorbance data, further detailed description will be omitted.

<Example of Operation of Blood Glucose Level Measuring Device 100>

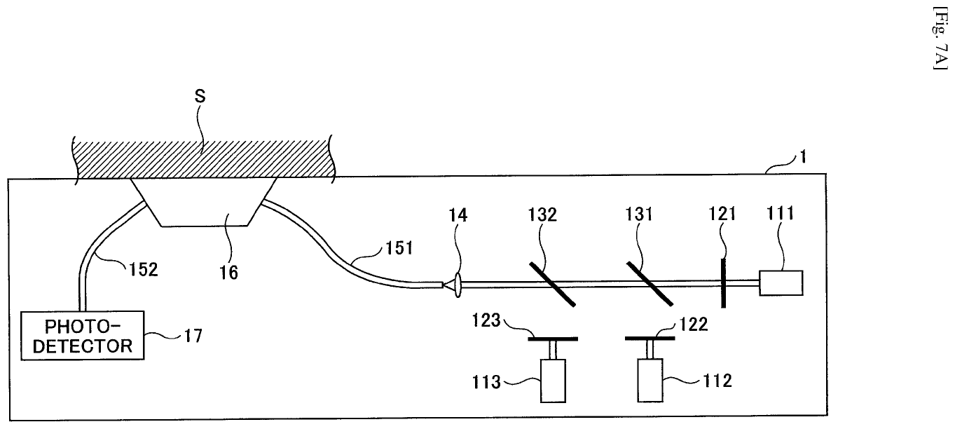

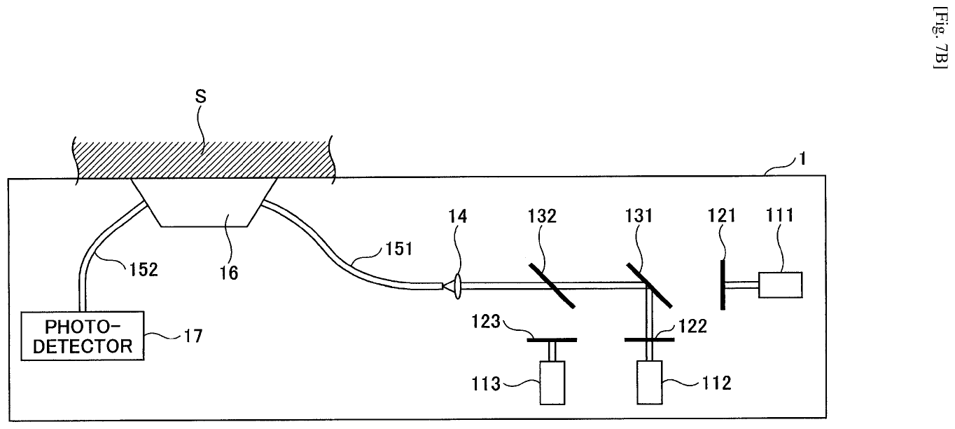

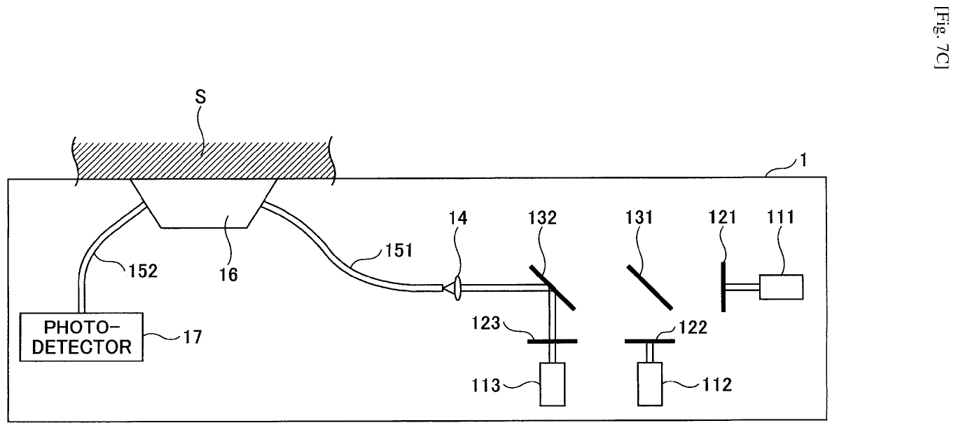

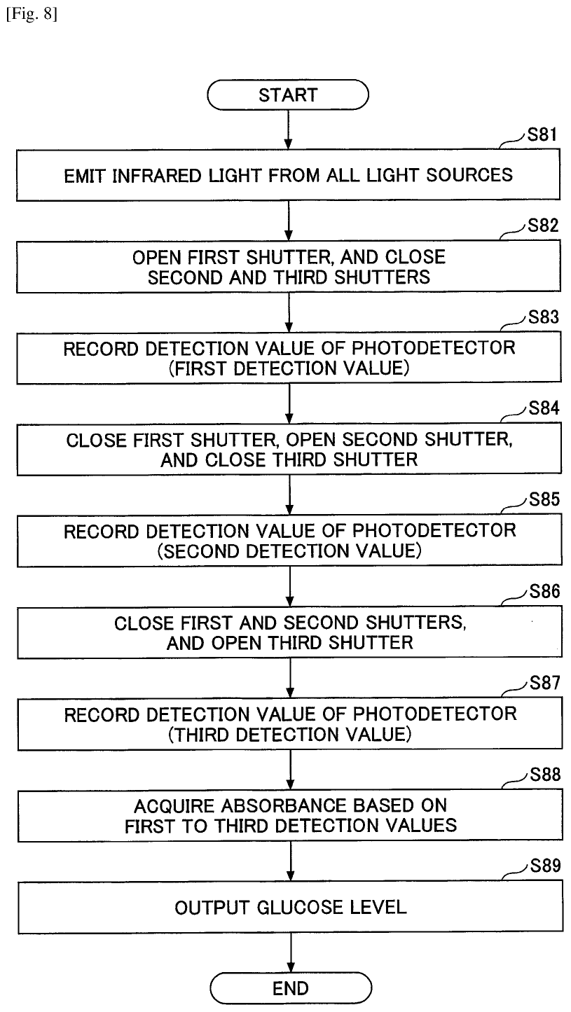

Next, an operation of the blood glucose level measuring device 100 will be described with reference to FIGS. 7A to 7C, and FIG. 8.

(Example of Switching Operation of Probe Light)

FIG. 7A is a diagram illustrating an example of a switching operation when first probe light is used; FIG. 7B is a diagram illustrating an example of a switching operation when second probe light is used; and FIG. 7C is a diagram illustrating an example of a switching operation when third probe light is used.

According to the embodiment, the incidence of probe light emitted by each of the light sources on the ATR prism 16 is controlled by opening and closing the respective shutters. The first light source 111, the second light source 112, and the third light source 113 emit infrared light at all times when measuring absorbance and a blood glucose level.

According to FIG. 7A, the first shutter 121 is open in response to a control signal. The first probe light emitted by the first light source 111 passes through the first shutter 121 and is transmitted through each of the first and second half mirrors 131 and 132, and then is guided to the first hollow optical fiber 151 via a coupling lens 14. Thereafter, the first probe light that has propagated through the first hollow optical fiber 151 enters the ATR prism 16.

Since the second shutter 122 and the third shutter 123 are each closed, the second probe light and the third probe light do not enter the ATR prism 16. Thus, in this state, the absorbance of the first probe light that is attenuated at the ATR prism 16 is measured.

According to FIG. 7B, the second shutter 122 is open in response to a control signal. The second probe light emitted by the second light source 112 passes through the second shutter 122, is reflected by the first half mirror 131. The reflected second probe light is transmitted through the second half mirror 132, and is guided to the first hollow optical fiber 151 via the coupling lens 14. Thereafter, the second probe light that has propagated through the first hollow optical fiber 151 enters the ATR prism 16.

By contrast, since the first shutter 121 and the third shutter 123 are each closed, the first probe light and the third probe light do not enter the ATR prism 16. Thus, in this state, the absorbance of the second probe light that is attenuated at the ATR prism 16 is measured.

In FIG. 7C, the third shutter 123 is open in response to a control signal. The third probe light emitted by the third light source 113 passes through the third shutter 123, is reflected by the second half mirror 132. The reflected third probe light is guided to the first hollow optical fiber 151 via a coupling lens 14. Thereafter, the third probe light that has propagated through the first hollow optical fiber 151 enters the ATR prism 16.

By contrast, since the first shutter 121 and the second shutter 122 are each closed, the first probe light and the second probe light do not enter the ATR prism 16. Thus, in this state, the absorbance of the third probe light that is attenuated at the ATR prism 16 is measured.

When all of the first shutter 121, the second shutter 122, and the third shutter 123 are closed, none of the first probe light, the second probe light, and the third probe light enters the ATR prism 16, and thus, the first probe light, the second probe light, and the third probe light do not reach the photodetector 17.

In this manner, the shutter controller 214 (see FIG. 6) acting as an incident light controller can control opening and closing of each of the shutters to switch between a state in which the first to third probe light sequentially enters the ATR prism 16 and a state in which one of the first to third probe light enters the ATR prism 16.

(Example of Operation of Blood Glucose Level Measuring Device 100)

FIG. 8 is a flowchart illustrating an example of an operation of the blood glucose level measuring device 100.

First, in step S81, in response to a control signal of the light source controller 212, all the first light sources 111, the second light sources 112, and the third light sources 113 emit infrared light. However, in this initial state, the first shutter 121, the second shutter 122, and the third shutter 123 are all closed.

Subsequently, in step S82, the shutter controller 214 opens the first shutter 121, and closes the second shutter 122 and the third shutter 123.

Subsequently, in step S83, the data recorder 216 records a detection value (a first detection value) of the photodetector 17 that is acquired by the light intensity acquisition unit 215.

Subsequently, in step S84, the shutter controller 214 opens the second shutter 122, and closes the first shutter 121 and the third shutter 123.

Subsequently, in step S85, the data recorder 216 records a detection value (a second detection value) of the photodetector 17 that is acquired by the light intensity acquisition unit 215.

Subsequently, in step S86, the shutter controller 214 opens the third shutter 123, and closes the first shutter 121 and the second shutter 122.

Subsequently, in step S87, the data recorder 216 records a detection value (a third detection value) of the photodetector 17 that is acquired by the light intensity acquisition unit 215.

Subsequently, in step S88, the absorbance output unit 217 acquires absorbance data of the first to third probe light based on the first to third detection values, and outputs the absorbance data to the biological information output unit 221.

Subsequently, in step S89, the biological information output unit 221 performs a predetermined calculation process based on the absorbance data of the first to third probe light, and acquires the blood glucose level data. The acquired blood glucose level data is output to a display 506 (see FIG. 5) for displaying the acquired blood glucose level data.

In this manner, the blood glucose level measuring device 100 can acquire and output blood glucose level data.

Note that the embodiment has illustrated an example in which the first shutter 121, the second shutter 122, and the third shutter 123, being electromagnetic shutters, are controlled to switch the incidence of the probe light on the ATR prism 16; however, switching of the incidence of the probe light on the ATR prism 16 is not limited to being controlled by the first shutter 121, the second shutter 122, and the third shutter 123. The incidence of the probe light on the ATR prism 16 may be switched between on (emission) and off (non-emission) of the plurality of light sources. A single light source configured to emit light of multiple wavelengths may be used to switch the light source on and off, on a per wavelength basis.

According to the embodiment, the first half-mirror and the second half-mirror are used as elements that transmit a portion of the probe light and reflect the remaining portion of the probe light. However, the present invention is not limited to this example. The elements that transmit a portion of the probe light and reflect a remaining portion of probe light may be a beam splitter, a polarizing beam splitter, or the like.

In addition, high refractive index materials, such as germanium, that transmit probe light have high surface reflectivity due to material characteristics. For example, when light polarized in the vertical direction (s-polarized) with respect to a plane direction of a substrate enters a substrate at an angle of incidence of 45 degrees, the ratio of transmission to reflection becomes approximately 1:1. This can be used to install a germanium plate at an angle of incidence of 45 degrees to replace the half mirror. The backside has 50% reflective component, so an anti-reflection coating is applied to the backside.

Since different types of variations may be applicable to the components according to the embodiment, such variations will be described as follows.

(Prevention of Adverse Effect of Linearity Error of Photodetector 17)

The photodetector 17 used in the blood glucose level measuring device 100 may contain a linearity error, and the linearity error of the photodetector 17 may cause a blood glucose level measurement error. To address this, the probe light intensity may be changed in three or more predetermined steps, and the probe light intensity and the detection value acquired by the photodetector 17 are compared in each of the steps to reduce an adverse effect of linearity error.

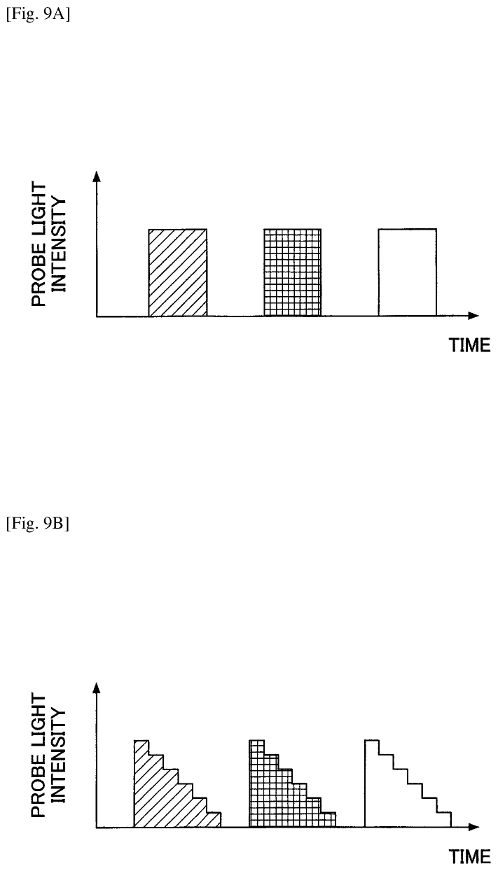

FIGS. 9A and 9B are graphs illustrating examples of probe light intensity that has changed in three or more steps as described above. FIG. 9A illustrates probe light intensity according to a comparative example, and FIG. 9B illustrates probe light intensity that has been changed in three or more steps. In FIGS. 9A and 9B, a portion indicated by diagonal hatching represents the first probe light intensity, a portion indicated by grid hatching represents the second probe light intensity, and a portion indicated by no hatching represents the third probe light intensity.

In FIG. 9A, the first, the second, and the third probe light intensities are all constant, whereas in FIG. 9B, the first, the second, and the third probe light intensities are gradually reduced in three or more steps. The intensities of the probe light can be changed to be emitted in three or more steps by changing the drive voltage or drive current of the light source in the predetermined three or more steps (six steps in the example of FIG. 9B). It should be noted that the light intensities in this case change in a period shorter than the switching control period of the probe light that is controlled by the shutter controller 214 (e.g., the period from steps S82 to S84 in FIG. 8).

When the photodetector 17 does not contain a linearity error, the detection value acquired by the photodetector 17 will vary linearly with the change in probe light intensity. However, when the photodetector 17 contains a linearity error, the detection value acquired by the photodetector 17 varies nonlinearly with the change in probe light intensity.

Accordingly, the probe light is emitted while changing the light intensity in three or more steps, intensity data of the emitted probe light and the detection value acquired by the photodetector 17 are compared at each of the steps, and a light intensity range within which linearity is ensured is specified. Of the probe light intensity that varies in three or more steps, only a portion of the probe light intensity where linearity is ensured is used to measure absorbance and blood glucose level. This reduces any adverse effect of the linearity error of the photodetector 17 in measuring absorbance and blood glucose levels.

Operations to specify the light intensity range within which linearity is ensured may be performed prior to blood glucose level measurement or simultaneously with blood glucose level measurement.

Further, since there is only one photodetector 17 with respect to a plurality of types of probe light, the process of reducing the adverse effect due to the linearity error of the photodetector 17 is not necessarily performed using all of the plurality of types of probe light, and instead may be performed using at least one type of probe light beams.

(Detection of Probe Light by Image Sensor)

The photodetector 17 is not limited to a photodetector that utilizes a single pixel (a light receiving element), and the photodetector 17 may instead be a linear image sensor in which pixels are linearly arranged or may be an area image sensor in which pixels are arranged in two dimensions.

It should be noted that a detection signal of the photodetector 17 is an integrated value of the received probe light intensity. Hence, when an optical path of incident light or emission light in the ATR prism 16 changes upon bringing the ATR prism 16 into contact with a living body S, the probe light intensities before and after the optical path change are integrated. This results in a detection error, and thus accurate absorbance data cannot be obtained.

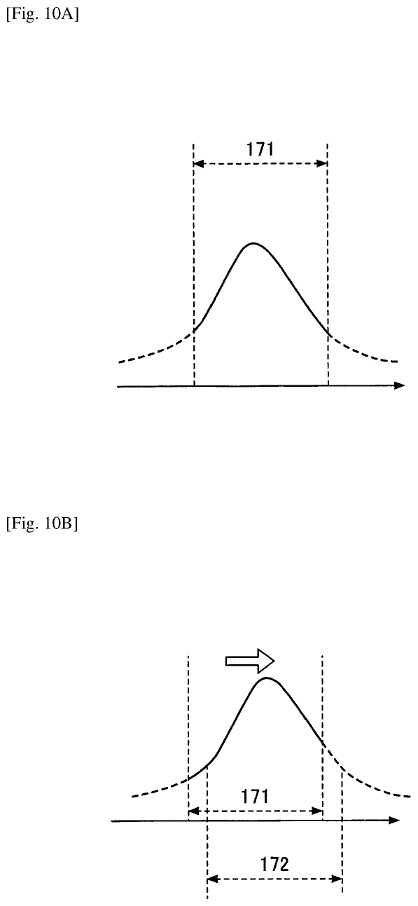

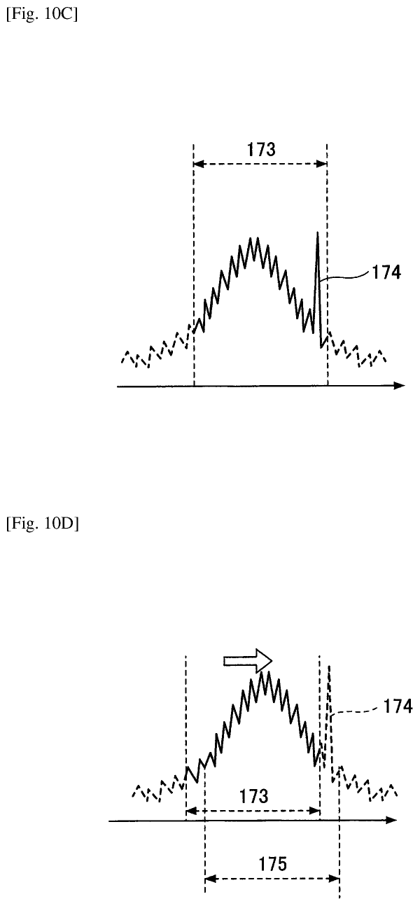

FIGS. 10A and 10B illustrate such a positional shift of the probe light, where a region 171 is a probe light receiving region of photodetector 17. As the probe light shifts in the direction indicated by an outlined arrow in FIG. 10B, a light intensity distribution of probe light in the region 171 changes, and in turn the detection signal acquired by the photodetector 17 changes.

By contrast, when an image sensor is used as the photodetector 17, a positionally shifted amount of the probe light is obtained from a probe light image captured by the image sensor. Thus, the integrated value of the light intensity distribution of the probe light after the positional shift may be used as a detection signal to correct any adverse effect due to the positional shift of the probe light. A region 172 of FIG. 10B illustrates a region where the integrated value of the light intensity distribution of the positionally shifted probe light is obtained.

Further, when coherent light, such as laser light, is used as the probe light, a fine spotty light intensity distribution called speckles may be superimposed onto the probe light. FIG. 10C illustrates an example of a cross-sectional light intensity distribution of probe light containing speckles. A reference numeral 174 indicates the singular point of the light intensity that may be included in a speckle image, and the singular point 174 is included in a region 173.

FIG. 10D illustrates a case where the probe light of FIG. 10C is positionally shifted in the direction indicated by a rightward outlined arrow. In this case, the singular point 174 is no longer included in the region 173, and the change in the detection signal before and after the positional shift becomes significant. Hence, any adverse effect of the positionally shifted probe light may be more preferably corrected by using the integrated value of the light intensity distribution as a detection signal in a region 175, in accordance with the positionally shifted amount of the probe light detected from the probe light image.

In addition, a contact region between the living body S and the ATR prism 16 is estimated, based on the probe light intensity distribution on the image sensor, and the detection value based on the detection signal of the image sensor is corrected in accordance with a sensitivity distribution within the ATR prism 16 plane. This can reduce variability errors in the measurement. Note that the sensitivity distribution within the ATR prism 16 plane has been acquired and stored before the start of the measurement.

(Incident Surface of Total Reflection Member)

According to the embodiment described above, the incident surface 161 of the ATR prism 16 is illustrated as a flat surface, but the incident surface 161 is not limited to the flat surface. The incident surface 161 of the ATR prism 16 may come in a variety of shapes, such as a surface having a diffusion face or a surface having a curvature.

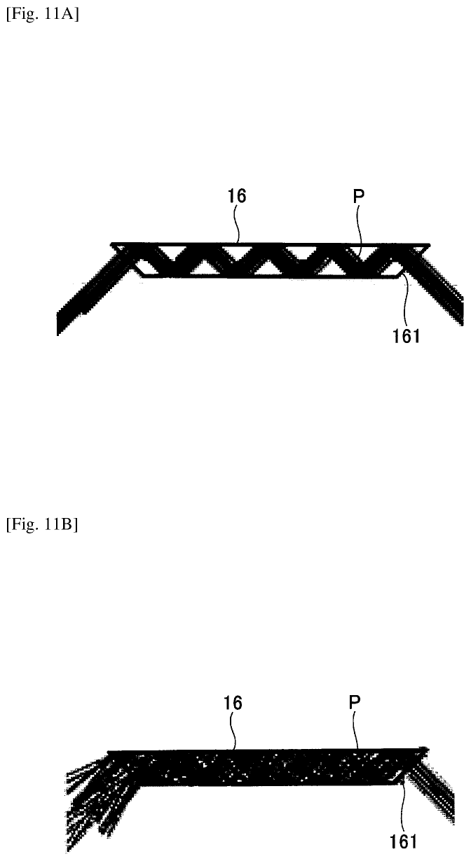

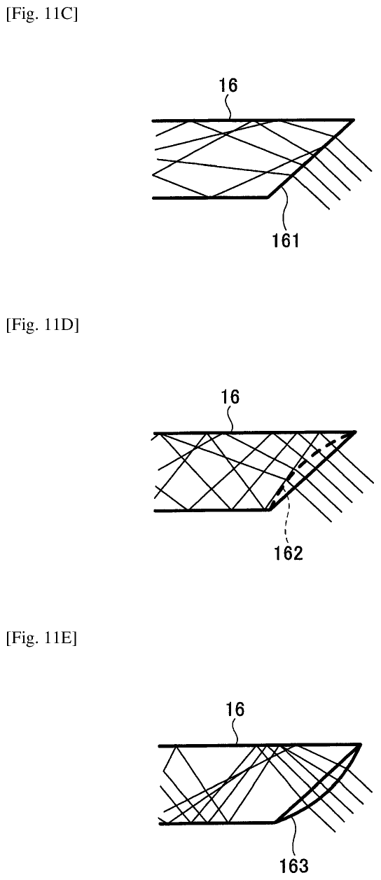

As illustrated in FIG. 11A, when the incident surface 161 is a flat surface, a propagating direction of the probe light within the ATR prism 16 becomes uniform in accordance with an angle of incidence on the incident surface 161. Accordingly, there may be a regional dependency of measurement sensitivity on a per region basis in the total reflection surface of the ATR prism 16 that is in contact with the living body S.

The detection signal of the photodetector 17 depends on a contact state, such as the size of a contact area of the living body S with respect to the ATR prism 16. Specifically, when the living body S, such as the lip or finger, is a subject to be measured, reproducibility of the contact state tends to be low, and variability in measurements may increase due to the regional dependency of the measurement sensitivity.

However, the use of a diffusion surface as the incident surface 161 randomly changes the propagating direction of the probe light in the ATR prism 16. This reduces the regional dependency of the measurement sensitivity, and also reduces variability in measurements, as illustrated in FIG. 11B.

In addition to the diffusion surface illustrated in FIG. 11B, another diffusion surface illustrated in FIG. 11C may be used. Further, the incident surface 161 can be a concave surface illustrated in FIG. 11D or a convex surface illustrated in FIG. 11E. The concave surface in FIG. 11D and the convex surface in FIG. 11E are examples of the incident surface having curvature. In this case, the optical path of the probe light can be changed as in the diffusion surface, and variability in measurements can be reduced by easing the regional dependency of the measurement sensitivity.

The same effect can be obtained by disposing a diffusing plate or a lens on the optical path at a position before the probe light enters the ATR prism 16. However, in this case, an increase in the number of device components may lead to an increase in the cost or a difference (machine difference) in the measurement values between the devices due to the assembly error. Hence, it is more preferable to apply a diffusion surface or curved surface to the incident surface 161 of the ATR prism 16 because the application of a diffusion surface or curved surface to the incident surface 161 can reduce a machine difference and keep costs from getting high.

(Light Guide Unit and Support Unit of Total Reflection Member)

When the first hollow optical fiber 151 and the second hollow optical fiber 152 are shifted relative to the ATR prism 16 in response the living body S contacting the ATR prism 16, incident efficiency and emission efficiency of the probe light with respect to the ATR prism 16 varies, and as a consequence variability in measurements may increase.

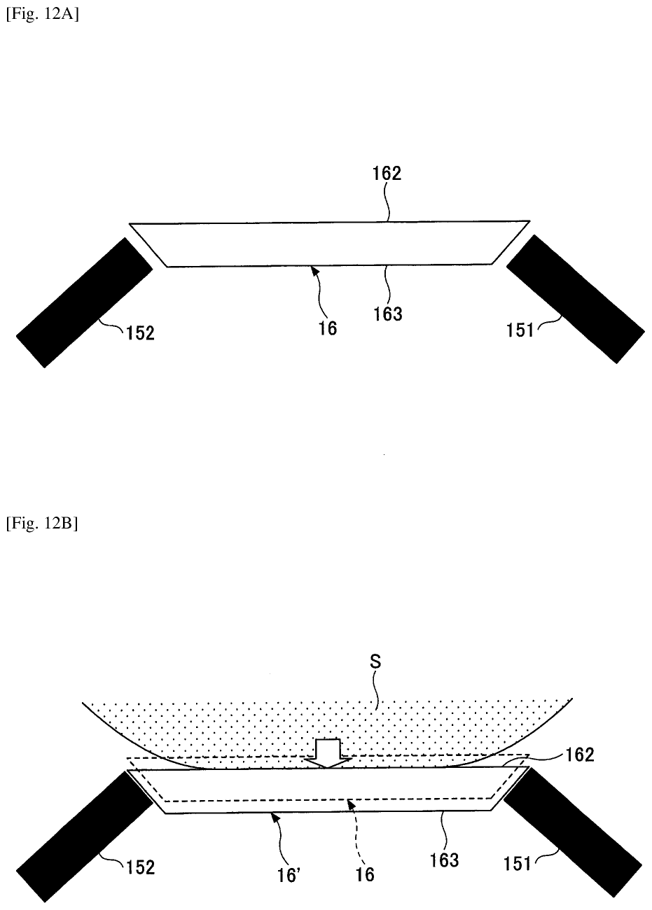

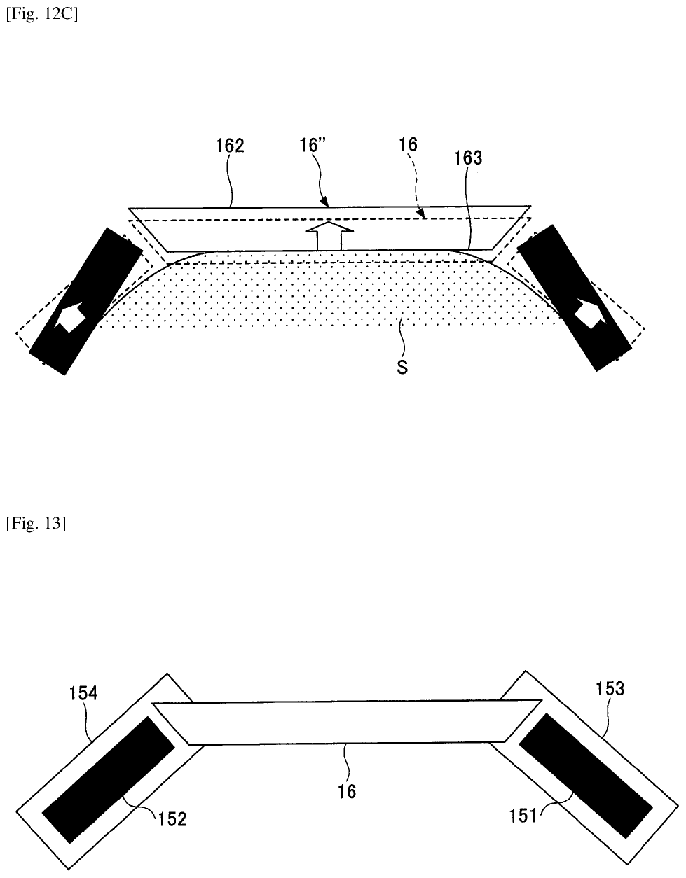

FIGS. 12A to 12C are diagrams illustrating relative positional shifts between such first and second hollow optical fibers 151 and 152 and an ATR prism 16. FIG. 12A illustrates a case where the ATR prism 16 is not in contact with the living body S. FIG. 12B illustrates a case where the living body S is in contact with the first total reflection surface 162 of the ATR prism 16. FIG. 12C illustrates a case where the living body S is in contact with the second total reflection surface 163 of the ATR prism 16.

As illustrated in FIG. 12B, when the living body S contacts the first total reflection surface 162 of the ATR prism 16, a pressure is applied downward as indicated by an outlined arrow, to cause the ATR prism 16 to shift downward. As a result, the ATR prism 16 is shifted to a position of an ATR prism 16′, and thus relative positions between the first and second hollow optical fibers 151 and 152 and the ATR prism 16′ change.

When the living body S contacts the second total reflection surface 163 of the ATR prism 16 as illustrated in FIG. 12C, a pressure is applied upward as indicated by an outlined arrow, to cause the ATR prism 16 to shift upward. As a result, the ATR prism 16 is shifted to a position of an ATR prism 16″, and thus relative positions between the first and second hollow optical fibers 151 and 152 and the ATR prism 16″ change.

Such relative positional shifts may cause fluctuation in the incident efficiency and emission efficiency of the probe light with respect to the ATR prism 16. Specifically, when a subject to be measured is a living body, it is not easy to maintain a constant contact pressure applied from the living body to the ATR prism 16. Hence, variability in measurements due to the relative positional shifts in particular tends to increase.

Accordingly, the first and second hollow optical fibers 151 and 152 and the ATR prism 16 are preferably supported by the same support member in order to reduce the relative positional shifts.

FIG. 13 is a diagram illustrating a configuration example of members that support a first hollow optical fiber 151, a second hollow optical fiber 152, and an ATR prism 16. A light guide support member 153 in FIG. 13 is a member that integrally supports the first hollow optical fiber 151 and the ATR prism 16. An emission support member 154 is also a member that integrally supports the second hollow optical fiber 152 and the ATR prism 16.

When the first hollow optical fiber 151 and the ATR prism 16 are integrally supported, and the living body S is in contact with the ATR prism 16, the two components (i.e., the first hollow optical fiber 151 and the ATR prism 16) move together. Therefore, relative positional shift does not occur between the first hollow optical fiber 151 and the ATR prism 16. Likewise, when the second hollow optical fiber 152 and the ATR prism 16 are integrally supported, and the living body S is in contact with the ATR prism 16, the two components (i.e., the second hollow optical fiber 152 and the ATR prism 16) move together. Therefore, relative positional shift does not occur between the second hollow optical fiber 152 and the ATR prism 16. Thus, fluctuation in the incident efficiency and the emission efficiency of the probe light caused by the contact between the living body S and the ATR prism 16 can be reduced, and thus variability in measurements can be reduced.

In the above example, the light guide support member 153 and the emission support member 154 are separate support members. However, the first hollow optical fiber 151, the second hollow optical fiber 152, and the ATR prism 16 may be supported by a single support member.

In addition, even in the case where a light guide unit is formed by an optical element such as a mirror or a lens, instead of using the first hollow optical fiber 151 as the light guide unit, the same advantageous effect as described above can be obtained by supporting the optical element and the ATR prism 16 integrally.

Further, in a manner similar to the light guide unit, the first light source 111, the second light source 112, the third light source 113, and the photodetector 17 may be integrally supported by the same support member, so that variability in measurements can be reduced.

(High Frequency Modulation of Light Source Drive Current)

When the probe light includes speckles, the detection value by the photodetector 17 may vary according to a speckle pattern thereby increasing variability in measurements. Since the speckles are generated by interference of the diffused probe light or the like, the generation of the speckles can be reduced by decreasing the coherence of the probe light. Hence, according to the embodiment, the coherence of the light source included in the blood glucose level measuring device can be reduced by superimposing the high frequency modulation component onto the current driving the light source. This can reduce variability in measurements of the absorbance caused by the speckles of the probe light.

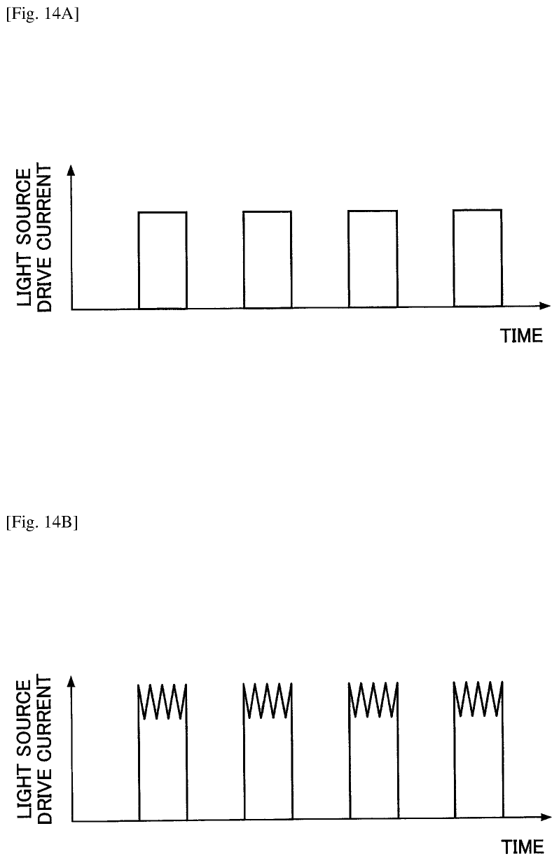

FIGS. 14A and 14B are graphs illustrating examples of a light source drive current. FIG. 14A illustrates a light source drive current according to a comparative example, and FIG. 14B illustrates a light source drive current with high frequency modulation.

The light source controller 212 (see FIG. 6) periodically outputs a pulsed drive current as illustrated in FIG. 14(a) to each of the first light source 111, the second light source 112, and the third light source 113 to emit pulsed probe light.

According to an embodiment, a high frequency modulation component is superimposed onto a pulsed drive current in FIG. 14A, and then the high frequency modulation component superimposed onto the pulsed drive current is output to the first light source 111, the second light source 112, and the third light source 113. The waveform of the high frequency modulation component may be sinusoidal or rectangular. The modulation frequency can be any modulation frequency from 1 MHz (megahertz) to several GHz (gigahertz).

By superimposing the high frequency modulation component onto a pulsed drive current, each of the first light source 111, the second light source 112, and the third light source 113 spuriously emits multimode laser light as probe light to reduce the coherence of the probe light. Accordingly, speckles of the probe light may be reduced by reducing coherence, and thus variability in measurements caused by the speckles may also be reduced.

### First Embodiment

Next, a blood glucose measuring device according to a first embodiment will be described.

The blood glucose level measuring device according to the first embodiment outputs blood glucose level information, based on light intensity of probe light reflected from the total reflection member with the total reflection member brought into contact with a subject to be measured. In addition, the blood glucose level measuring device displays information relating to the light intensity and absorbance of the probe light, and on a pressure of the subject to be measured with respect to the total internal reflection member during measurement, or the blood glucose level measuring device displays information relating to a contact region between the subject to be measured and the total reflection surface of the total reflection member. The information relating to the contact region is generated based on a contact image between the total reflection member and the subject to be measured.

Light intensity of the probe light, or absorbance varies with a contact region of the subject to be measured with respect to the total reflection member. The absorbance is a calculated value of the light intensity before and after the subject is in contact with the total reflection member. One of the factors for this is that as the size of the contact area increases, the size of the area in which the subject to be measured contacts a penetration occurring region will increase at the interface of the total reflection member, and more light will be absorbed.

Further, a penetration depth will fail to be uniform depending on the position of the total reflection member in the following cases:

when the two opposing surfaces of the total reflection member are not strictly parallel, but the angle of the probe light with respect to the total reflection member changes at the time of incidence and at the time of emission; and

when the probe light propagating in the total reflection member is not strictly parallel but diffuses.

Thus, in order to estimate blood glucose levels more accurately, it is desirable to measure not only the size of the contact area but also the region in which the subject contacts the total reflection member in a consistent manner, in view of the reproducibility of measurement.

The light intensity and absorbance of the probe light also vary depending on the pressure (contact pressure) of the subject with respect to the total reflection member.

This may be because, when the subject is an elastic body such as a living body (e.g., lip, finger, etc.), the size of the contact region changes due to the contact pressure, and the internal composition of the subject changes due to changes in the contact pressure, which affects the flow of glucose-containing body fluids such as blood or interstitial fluid.

Accordingly, it is preferable to measure the contact pressure in a consistent manner.

Since the light intensity or absorbance of the probe light varies with a contact pressure and a contact region of the subject to be measured with respect to the total reflection member, the light intensity or absorbance of the probe light may be considered as an index making the contact region or the contact pressure described above consistent. However, even if the contact pressure or contact region is the same, the light intensity and absorbance of the probe light change with time immediately after the subject to be measured comes into contact with the total reflection member. This is considered to be due to the fact that body fluid flows under pressure and the internal composition of the subject to be measured changes even if there is no change in contact pressure, or the like. Accordingly, the data recorded at the time at which the light intensity has converged sufficiently can be used as data for estimating the blood glucose level, thereby increasing the accuracy of the estimation.

In addition, when the subject to be measured is a lip, for example, the contact pressure of the subject with respect to the total reflection member is often weak in the first place. Consequently, the signal strength to be detected is small, and the detection accuracy of the pressure may often be insufficient. In addition, in terms of the contact region, it is not preferable to dispose a contact sensor directly between the subject to be measured and the total reflection member, on the basis of the measuring principle of using the penetration light from the total reflection member. In addition, even if the contact region is estimated indirectly by, for example, a camera, the accuracy of estimating the contact region is often insufficient.

The reproducibility of the measurement may be improved by displaying the contact pressure or contact region such that when the subject can adjust the contact region using the displayed contact pressure or contact region as an index. However, from the above viewpoint, adjusting the light intensity or absorbance of the probe light to an index allows for further improvement in resolution or reproducibility, thereby providing a significant effect on the accuracy in estimating the blood glucose levels.

The light intensity of the probe light or the resolution of the absorbance is determined by the performance of the A/D converter that converts the analog signal from the photodetector into a digital signal. However, the resolution or precision of the signal is often high relative to the resolution or precision of the contact pressure or contact region.

According to the present embodiment, the user of the blood glucose measuring device adjusts how he contacts his lip with the total reflection member while visually viewing information about the probe light intensity, absorbance, contact pressure, or contact region. This allows the blood glucose measuring device to accurately measure blood glucose levels, with contact state fluctuation between the subject and the total reflection member being reduced. Note that according to the present embodiment, all the information relating to the probe light intensity, absorbance, contact pressure, or contact region is displayed as an example, but information relating to any one of these may be displayed.

Further, the user of the blood glucose level measuring device may include patients who are subject to blood glucose level measurement, and physicians and nurses who operate the blood glucose level measuring device. Hereinafter, an example will be described in which the user is a subject.

<Example of Configuration of Blood Glucose Level Measuring Device 100a>

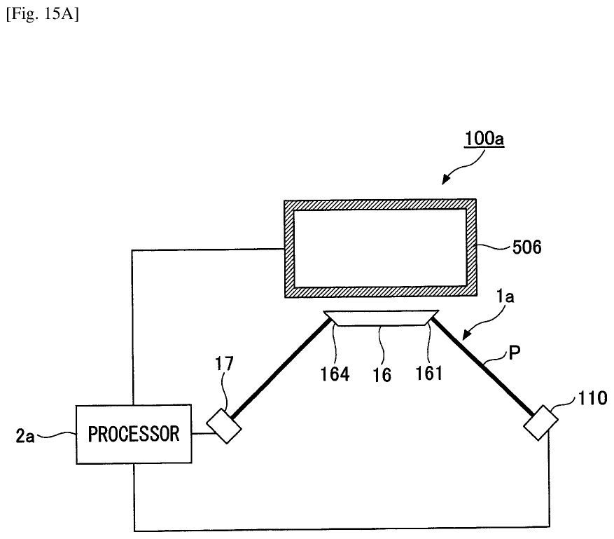

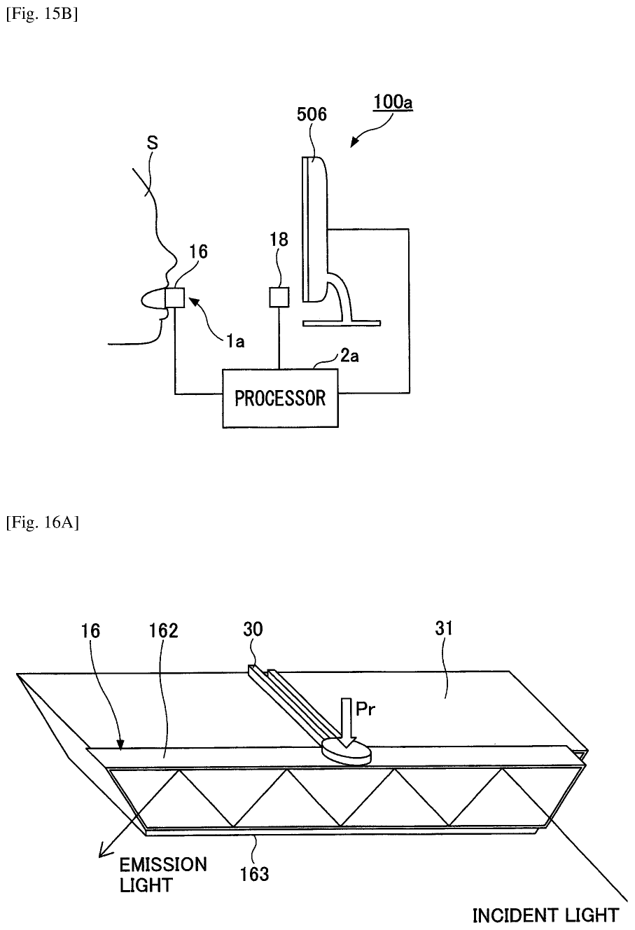

First, a configuration of a blood glucose level measuring device 100a according to the first embodiment will be described. FIGS. 15A and 15B are diagrams illustrating an example of the configuration of the blood glucose level measuring device 100a. FIG. 15A is a diagram illustrating a configuration of a measuring unit 1a, and FIG. 15B is a diagram illustrating arrangement of the measuring unit 1a, the camera 18, and the display 506.

As illustrated in FIGS. 15A and 15B, the blood glucose level measuring device 100a includes the measuring unit 1a, the processor 2a, and the camera 18.

The measuring unit 1a includes an infrared light source unit 110.

The infrared light source unit 110 may include a plurality of light sources where probe light is switched by respective shutters, as illustrated in the overall configuration example in FIG. 1. However, the configuration is not limited to this example. The infrared light source unit 110 may be a continuous spectrum light source including light of various wavelengths or a variable wavelength light source when the light includes wavelengths in the infrared region used for estimation of blood glucose. In such a case, the details of the absorbance calculation method in the processor are different. However, in the case of a continuous spectrum light source, for example, the absorbance of the probe light is calculated using the operation generally used in Fourier transform infrared spectroscopy.

In the following, the infrared light source unit 110 is an example of a variable wavelength quantum cascade laser. The infrared light source unit 110 includes a first probe light configured to emit laser light of 1050 cm⁻¹, a second probe light configured to emit laser light of 1070 cm⁻¹ as, and a third probe light configured to emit laser light of 1100 cm⁻¹.

In other words, the infrared light source unit 110 includes respective functions of the first light source 111, the second light source 112, and the third light source 113, according to the embodiment described above (see FIG. 1). According to the first embodiment, emission of the first to third probe light by the infrared light source unit 110 can be switched by a control signal. Thus, illustration of a configuration for switching the wavelengths of the first shutter 121, the second shutter 122, the third shutter 123, the first half mirror 131, and the second half mirror 132 in FIG. 1 will be omitted. Hereinafter, the first to third probe light is collectively referred to as a probe light P, unless otherwise specified.

Probe light P emitted from the infrared light source unit 110 enters ATR prism 16 via the incident surface 161, the entered probe light P is attenuated by the living body S that is in contact with ATR prism 16, and the attenuated probe light P is then emitted from ATR prism 16 via an emission surface 164. The probe light P emitted from the ATR prism 16 reaches the photodetector 17, where light intensity of the probe light P is detected.

FIG. 15A illustrates an example in which probe light P from the infrared light source unit 110 directly enters the ATR prism 16. However, the probe light P may be configured to enter the ATR prism 16 through the first hollow optical fiber 151 as illustrated in FIG. 1. FIG. 15A also illustrates an example in which the probe light P from the ATR prism 16 directly enters the photodetector 17. However, the probe light P may be configured to enter the photodetector 17 through the second hollow optical fiber 152 as illustrated in FIG. 1.

As illustrated in FIG. 15B, the processor 2a is electrically coupled to the measuring unit 1a and the camera 18, so as to cause the display 506 to visually display, to the subject, various types of information based on the light intensity, absorbance, and the later-described contact pressure acquired by the measuring unit 1a, and an image captured by the camera 18. In this case, the camera 18 is an example of an imaging unit configured to capture an image of a vicinity of a contact region between the total reflection surface of the total reflection member and the subject to be measured.

The measurement by the blood glucose level measuring device 100a is performed in a state where a subject holds the ATR prism 16 of the measuring unit 1a in his mouth so that the subject's lip is in contact with the total reflection surface of the ATR prism 16. In this state, the subject who holds the ATR prism 16 in his mouth is able to adjust a contact state between the subject's lip and a total reflection surface of the ATR prism 16 while viewing various types of information displayed on the display 506.

Next, a pressure sensor 30 (an example of a pressure detector) disposed on the ATR prism 16 will be described. FIG. 16A is a diagram illustrating a configuration example where one pressure sensor 30 is disposed. FIG. 16B is a diagram illustrating a configuration example where two pressure sensors 30 are disposed at respective opposite ends of the ATR prism 16. FIG. 16C is a diagram illustrating a configuration example where pressure sensors 30 (three in this case) are disposed.

As illustrated in FIGS. 16A to 16C, a total reflection support 31 is a member configured to contact one side of the ATR prism 16 (other than the probe light incident and emission surfaces) to support the ATR prism 16, or to support the pressure sensor 30 disposed on the first total reflection surface 162.

The pressure sensor 30 is fixed by adhesion or the like in contact with at least one of the ATR prism 16 or the pressure sensor 30. The pressure sensor 30 is a sensor configured to detect the contact pressure received by the ATR prism 16 from the lip when the subject holds the ATR prism 16 in his mouth. Various types of pressure sensors may be applied as the pressure sensor 30; examples include capacitive sensors, strain gauge sensors, pressure-sensitive resistance sensors whose resistance varies with pressure, and pressure sensors utilizing MEMS technology.

FIGS. 16A to 16C illustrate examples in which the pressure sensor 30 is disposed only on the first total reflection surface 162 of the ATR prism 16; however, the pressure sensor 30 may be disposed on at least one of the first total reflection surface 162 or the second total reflection surface 163 of the ATR prism 16.

Note that a contact pressure on the prism near the two ends of the lip is easily fluctuated because it is relatively difficult for the user to apply force to the prism with the two ends of the lip, or there is an individual difference in the size of the mouth. Hence, as illustrated in FIG. 16B, two pressure sensors 30 are disposed near respective opposite ends of the ATR prism 16 so as to detect contact pressure near the two ends of the lip. Further, as illustrated in FIG. 16C, three pressure sensors 30 may be disposed so as to detect a distribution of contact pressure.

When the pressure sensor 30 is disposed on the total reflection surface, a field penetration from the total reflection surface does not occur in a region where the pressure sensor 30 is disposed, and an attenuation action by the living body S is no longer obtained. As a result, the region where the pressure sensor 30 is disposed does not serve as a measurement sensitivity region.

Accordingly, by disposing the pressure sensor 30 in a region where the contact region easily fluctuates, such as near two opposite ends of the ATR prism 16, variability can be reduced in absorbance measurements by contact region fluctuation.

However, when the pressure sensors 30 are disposed in all the regions where total reflection occurs in the ATR prism 16, measurement based on the ATR method is not possible. Hence, it is preferable not to dispose the pressure sensor 30 in at least a part of the regions where total reflection occurs so as to secure a measurement sensitivity region.

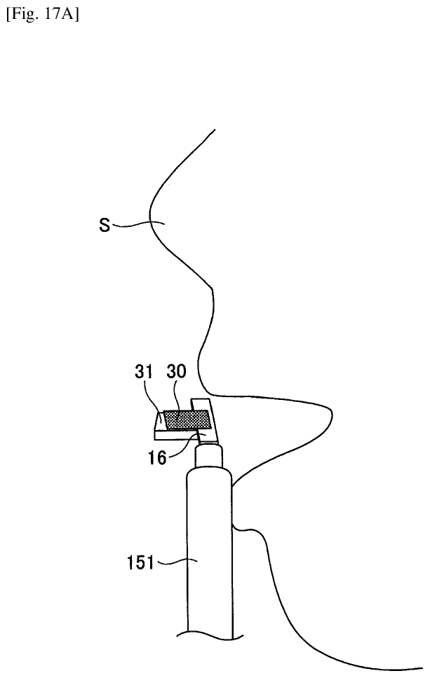

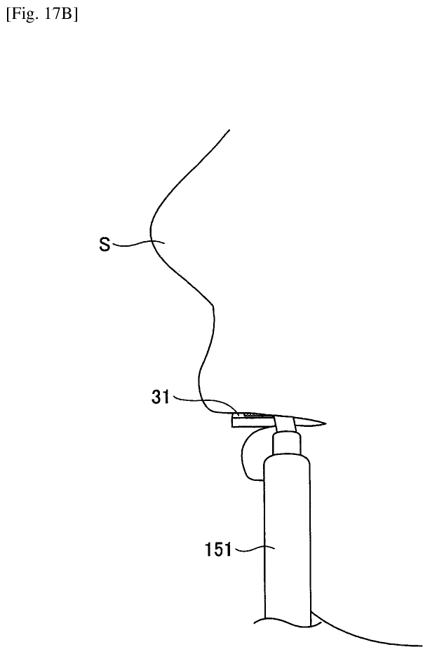

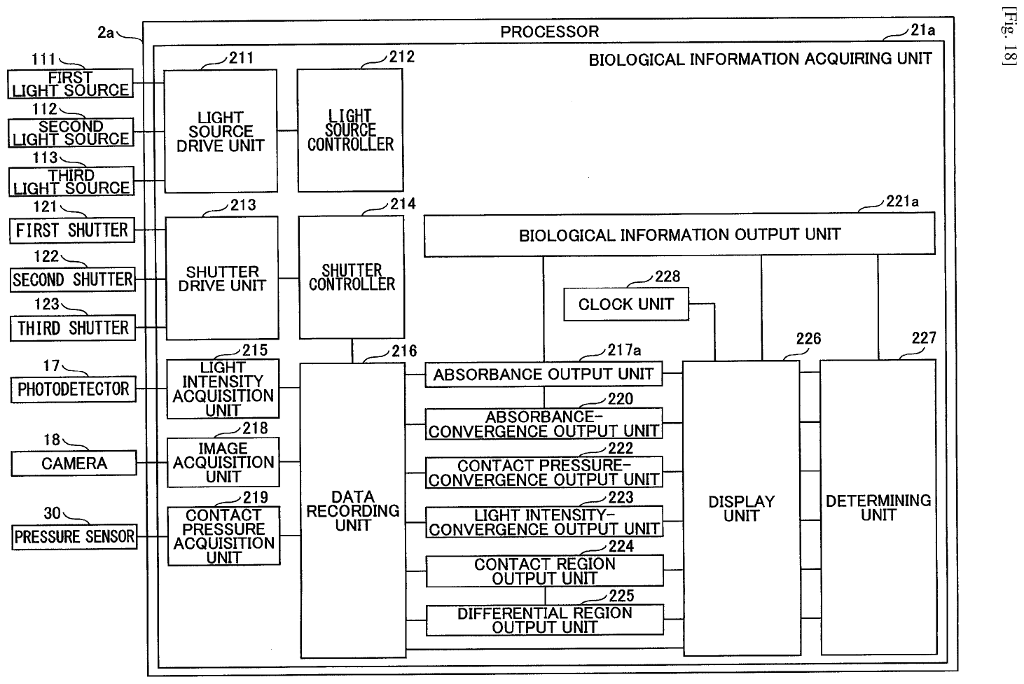

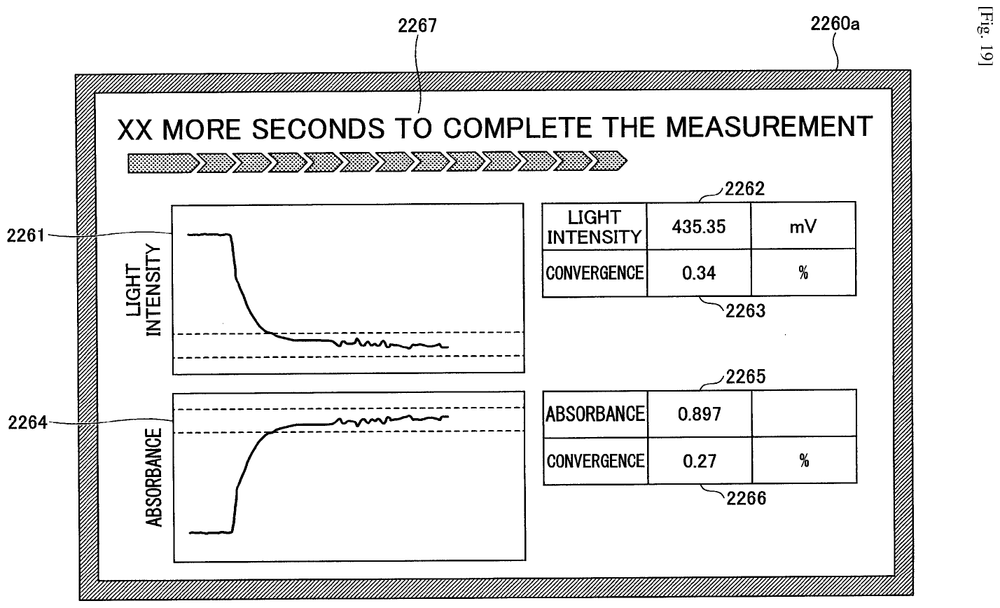

FIGS. 17A and 17B are diagrams illustrating an example of an arrangement of the ATR prism 16 and the pressure sensor 30 with respect to the lip. FIG. 17A illustrates an arrangement indicating where the ATR prism 16 and the pressure sensor 30 are before contact with the lip and FIG. 17B illustrates an arrangement indicating where a person holds the ATR prism 16 in his mouth.

As can be seen in FIGS. 17A and 17B, the size of the ATR prism 16 relative to the subject's lip is small. As a result, when the subject holds the ATR prism 16 in his mouth, the lip is in contact with both the ATR prism 16 and the total reflection support 31. Accordingly, although FIGS. 17A and 17B illustrate an example in which the pressure sensor 30 is disposed on both the total reflection surface and the total reflection support 31 of the ATR prism 16, the pressure sensor 30 may be disposed and fixed to only the total reflection support 31.

<Example of Function Configuration of the Processor 2a>

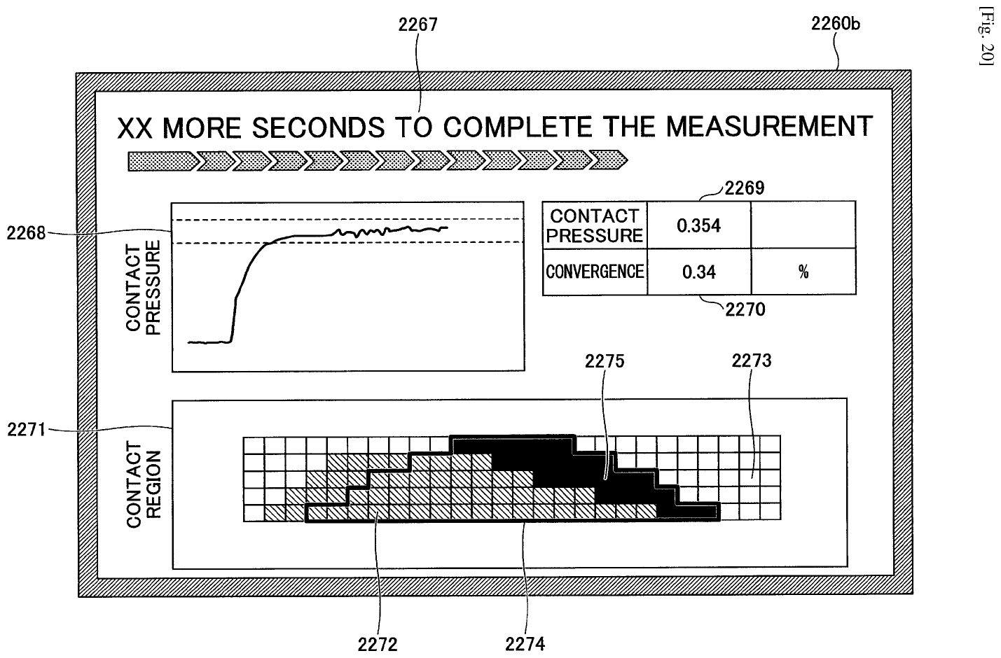

Next, a functional configuration of the processor 2a will be described. FIG. 18 is a block diagram illustrating a functional configuration example of a processor 2 according to the first embodiment. As illustrated in FIG. 18, the processor 2a includes a biological information acquisition unit 21a.

The biological information acquisition unit 21a includes an image acquisition unit 218, a contact pressure acquisition unit 219, an absorbance output unit 217a, an absorbance-convergence output unit 220, a contact pressure-convergence output unit 222, a light intensity-convergence output unit 223, a contact region output unit 224, a differential region output unit 225, a display unit 226, a determining unit 227, a biological information output unit 221a, and a clock unit 228.

Of these, a function of the image acquisition unit 218 is provided by a camera I/F 520 or the like, a function of the contact pressure acquisition unit 219 is provided by a pressure detection I/F 521 or the like, and a function of the display unit 226 is provided by a display 506 or the like. Respective functions of the absorbance output unit 217a, the absorbance-convergence output unit 220, the contact pressure-convergence output unit 222, the light intensity-convergence output unit 223, the contact region output unit 224, the differential region output unit 225, the determining unit 227, and the biological information output unit 221a are implemented by causing the CPU 501 or the like to execute a predetermined program. The clock unit 228 is implemented by counting the clock of the CPU 501 or the like.

The image acquisition unit 218 acquires a contact image of the subject's lip that is in contact with the ATR prism 16. The contact image of the subject's lip that is in contact with the ATR prism 16 is continuously output by the camera 18 in a predetermined period. The image acquisition unit 218 outputs the acquire contact image to the data recorder 216. The data recorder 216 records this contact image.

The contact pressure acquisition unit 219 acquires contact pressure data (pressure) of the subject's lip in contact with the ATR prism 16 by sampling a detection signal. The detection signa is continuously output by the pressure sensor 30 in a predetermined period. The contact pressure acquisition unit 219 outputs the acquired contact pressure data to the data recorder 216. The data recorder 216 records the contact pressure data. The contact pressure data may be the mean of the contact pressures that are sampled during a predetermined period.

The absorbance output unit 217a outputs absorbance data (absorbance) acquired by calculation based on the detection value read from the data recorder 216 to each of the absorbance-convergence output unit 220, the biological information output unit 221a, and the display unit 226.

The absorbance-convergence output unit 220 outputs the absorbance-convergence data (absorbance-convergence) acquired by calculation based on the absorbance data to the display unit 226. Here, the absorbance-convergence indicates the ratio of the absorbance fluctuation range to the mean of absorbance in a predetermined period of time, and represents the stability of the absorbance to be acquired. The absorbance fluctuation range is calculated by the standard deviation of the absorbance in the predetermined period of time. The same applies to a fluctuation range of the contact pressure, and the like noted below.

The contact pressure-convergence output unit 222 (the pressure-convergence output unit) outputs the contact convergence data (the pressure-convergence) to the display unit 226. The contact convergence data is acquired by calculation based on the contact pressure data read from the data storage unit 216. Here, the contact pressure-convergence indicates the ratio of the contact pressure fluctuation range to the mean of the contact pressure in a predetermined period of time, and represents the stability of the contact pressure to be acquired.

The light intensity-convergence output unit 223 outputs, to the display unit 226, light intensity-convergence data (light intensity-convergence). The light intensity-convergence data is acquired by calculation based on the detection value of light intensity read from the data recorder 216. Here, the light intensity-convergence is the ratio of the light intensity fluctuation range to the mean of light intensity in a predetermined period of time. The light intensity-convergence represents a value of the stability of the light intensity to be acquired.

The contact region output unit 224 outputs contact region data (contact region) between the lip of the subject and the total reflection surface of the ATR prism 16 to each of the differential region output unit 225 and the display unit 226. The contact region data is acquired by calculation based on the contact image read from the data recorder 216.

The differential region output unit 225 calculates differential region data (differential region) between the contact region and a predetermined target contact region, and outputs the differential region data to the display unit 226.

The display unit 226 displays, on the display 506, the absorbance data, absorbance-convergence data, contact pressure-convergence data, light intensity-convergence data, contact region data, and differential region data.

The display unit 226 displays, on the display 506, each of the contact pressure data read from the data recorder 216, light intensity data blood glucose level data input from the biological information output unit 221, and information indicating the time remaining until the end of the measurement that is input from the clock unit 228.

Further, the display unit 226 outputs, to the determining unit 227, the absorbance data, absorbance-convergence data, contact pressure data, contact pressure-convergence data, light intensity data, light intensity-convergence data, contact region data, and differential region data.

The determining unit 227 makes a determination to start acquiring a blood glucose level, the determination being made based on at least one of light intensity-convergence data, absorbance-convergence data, or contact convergence data, and based on a combination of contact pressure data and contact region data. The determining unit 227 then outputs the determination result to the biological information output unit 221.

Specifically, the determining unit 227 makes a determination to start acquiring a blood glucose level, and outputs the determination result to the biological information output unit 221 when the contact pressure data Pᵣ is within a predetermined contact pressure range (pressure range), and the contact region data A is within a predetermined contact region range; and when at least one of the following conditions a) to c) is satisfied:
a) the light intensity-convergence data I_{c} is not more than a predetermined light intensity threshold value I_{cth},
b) the absorbance-convergence data K_{c} is not more than a predetermined absorbance threshold value K_{cth}, and
c) the contact pressure-convergence data P_{c} is not more than a predetermined contact pressure threshold value P_{cth}.

According to the first embodiment, the determining unit 227 makes a determination to start acquiring a blood glucose level, and also output the determination result to the biological information output unit 221 when the contact pressure data Pᵣ is within a predetermined contact pressure range (pressure range), the contact region data A is within a predetermined contact region range, the light intensity data I is within a predetermined light intensity range, the light intensity-convergence data I_{c} is not more than a predetermined light intensity threshold value I_{cth}, the absorbance data K is within a predetermined absorbance range, the absorbance-convergence data K_{c} is not more than a predetermined absorbance threshold value K_{cth}, and the contact pressure-convergence data P_{c} is not more than a predetermined contact pressure threshold value P_{cth}.

Herein, the term “within a predetermined light intensity range” means that the light intensity data I is within a range between the minimum light intensity Iₘᵢₙ or more and the maximum light intensity Iₘₐₓ or less, and the term “within a predetermined contact pressure range” means that the contact pressure data Pᵣ is within a range between the contact pressure minimum Pₘᵢₙ or more and the contact pressure maximum Pₘₐₓ or less. In addition, the term “within a predetermined absorbance range” means that an absorbance data K is within a range between the absorbance minimum value Kₘᵢₙ or more and the absorbance maximum Kₘₐₓ or less, and the term “a predetermined contact region range” means that a contact region data (contact area) A is within a range between the contact region minimum value Aₘᵢₙ or more and the contact region maximum value Aₘₐₓ or less.

When the determining unit 227 makes a determination to start acquiring a blood glucose level, the biological information output unit 221a outputs to the display unit 226 the blood glucose level data acquired by calculation based on the absorbance data input from the absorbance output unit 217.

The clock unit 228 outputs a remaining time to the end of the measurement to the display unit 226, where the remaining time is acquired based on the predetermined measurement time and the time at which the measurement has been started.

<Example of Display Screen by Display Unit 226>

Next, a display screen by the display unit 226 will be described with reference to FIGS. 19 and 20. FIG. 19 is a diagram illustrating an example of a display screen that displays light intensity and absorbance, and FIG. 20 is a diagram illustrating an example of a display screen that displays a contact pressure and a contact region.

As illustrated in FIG. 19, a display screen 2260a displays a light intensity graph 2261 representing a light intensity change over time, and also displays light intensity information 2262 and light intensity-convergence information 2263, which are illustrated on the right-hand side of the light intensity graph 2261.

In addition, the display screen 2260a displays an absorbance graph 2264 representing an absorbance change over time, and also displays absorbance information 2265 and absorbance-convergence information 2266 on the right-hand side of the absorbance graph 2264.

According to the light intensity graph 2261 illustrated in FIG. 19, at an early stage (left-hand side of the graph), the ATR prism 16 is yet to be in contact with the subject's lip, so that ambient light and the like are also incident on the photodetector 17. Thus, the light intensity is greater at the early stage (left-hand side of the graph) by the amount corresponding to the ambient light and the like. Subsequently, the output of light intensity is significantly reduced due to blockage of the ambient light, and the like at the timing of the ATR prism 16 and the subject's lip being in contact with each other. Thereafter, the light intensity is gradually reduced, and then stabilized.

According to the absorbance graph 2264 illustrated in FIG. 20, the absorbance is small because less probe light enters the photodetector 17 during the period when the ATR prism 16 and the subject's lip are not in contact yet. Subsequently, when the ATR prism 16 contacted the subject's lip, the probe light increased and the absorbance greatly increased. Absorbance then gradually increased and then stabilized.

Remaining time information 2267 to the end of the measurement is displayed at an upper part of the display screen 2260a. This time is displayed to count down over time.

As illustrated in FIG. 20, a display screen 2260b displays a contact pressure graph 2268 representing a change in the contact pressure over time, and also displays the contact pressure information 2269 and the contact pressure-convergence information 2270 on the right-hand side of the contact pressure graph 2268.

A contact region map 2271 at a lower part of the display screen 2260b is a map representing a contact region between the subject's lip and the total reflection surface of the ATR prism 16. The grid in the contact region map 2271 represents the pixels of the map.

Within the contact region map 2271, a contact region information 2272 depicted by diagonally hatched pixels represents a region where the total reflection surface of the ATR prism 16 is in contact with the subject's lip. A non-contact region information 2273 depicted by white pixels, represents a region where the total reflection surface of the ATR prism 16 is not in contact with the subject's lip.

A target contact region information 2274, which is enclosed by a thick solid line, is an ideal contact region between the subject's lip and the total reflection surface of the ATR prism 16. This ideal contact region is predetermined. The differential region information 2275 filled in black is a region representing differentials between the contact region information 2272 and the target contact region information 2274. The differential region information 2275 represents a deviation of the contact region information 2272 from the target contact region information 2274.

The contact region information 2272 is generated based on an image captured by the camera 18. The image is captured from the front side of a subject who holds the ATR prism 16 in his mouth. Specifically, the contact image is processed to detect the presence or absence of a gap between the total reflection surface and the lip. An image region corresponding to a lip portion where a gap is detected is displayed as non-contact region information 2273. An image region corresponding to a lip portion where a gap is not detected is displayed as contact region information 2272. Information about a depth direction (a vertical direction of the contact region in the contact region map 2271 of FIG. 20) is displayed as the contact region information 2272 in accordance with a predetermined shape of the lip.

The display screen 2260a and the display screen 2260b may be displayed on the display 506 simultaneously, or either of the display screen 2260a and the display screen 2260b may be switchably displayed.

Numerical data such as a graph, a map, and light intensity on the display screen 2260a and 2260b are updated with a predetermined period, so that the latest information is displayed in real time. As a contact state between the ATR prism 16 and the subject's lip varies, the light intensity, absorbance, and contact pressure graphs and numerical values, as well as the contact region map, change according to the contact state.

The subject can adjust a holding fashion of the ATR prism 16 in his mouth while viewing the information displayed on the display screens 2260a and 2260b. The subject can thus adjust the contact between his lip and the total reflection surface of the ATR prism 16 to stabilize the graph, numerical values, and contact region map displayed on the display screens 2260a and 2260b.

As a result of adjustment made by the subject, the determining unit 227 makes a determination to start acquiring a blood glucose level when the contact pressure data Pᵣ is within the predetermined contact pressure range, the contact region data A is within the predetermined contact region range, the light intensity-convergence data I_{c} is the predetermined light intensity threshold I_{cth} or less, the absorbance-convergence data K_{c} is the predetermined absorbance threshold K_{cth} or less, and the contact pressure-convergence data P_{c} is the predetermined contact pressure threshold P_{cth} or less. In other words, the determining unit 227 makes a determination to start acquiring a blood glucose level at timing where the ATR prism 16 held in the subject's mouth becomes in a predetermined stabilized state. In response to this determination, the biological information output unit 221 outputs, to the display unit 226, a blood glucose level acquired by calculation based on the absorbance input from the absorbance output unit 217a at that timing where the ATR prism 16 held in the subject's mouth becomes in the predetermined stabilized state.

<Example of Process by Processor 2a>

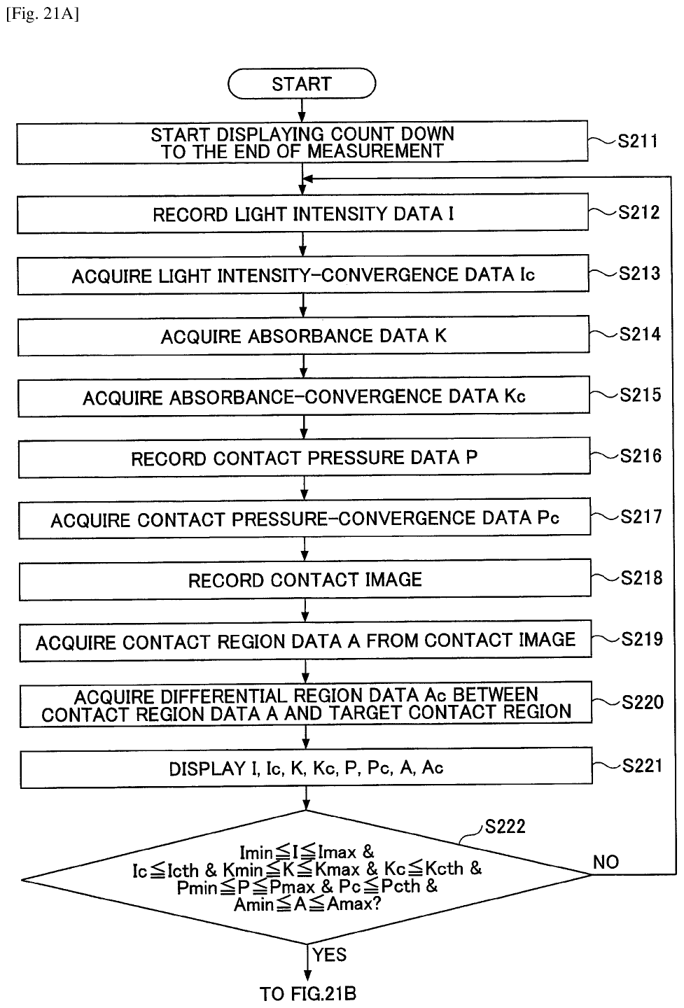

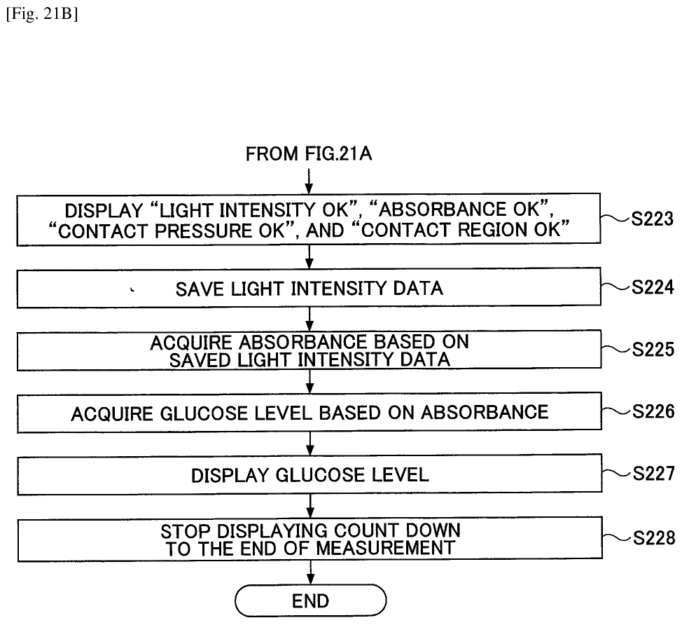

Next, a process performed by the processor 2a will be described. FIGS. 21A and 21B illustrate a flowchart of the processing performed by the processor 2a.

First, in step S211, the display unit 226 starts displaying time information to the end of the measurement that is input from the clock unit 228.

Subsequently, in step S212, the data recorder 216 records light intensity data I acquired by the light intensity acquisition unit 215.

Subsequently, in step S213, the light intensity-convergence output unit 223 acquires light intensity-convergence data I_{c} and outputs the light intensity-convergence data I_{c} to the display unit 226.

Subsequently, in step S214, the absorbance output unit 217a acquires absorbance data K and outputs the absorbance data K to the display unit 226.

Subsequently, in step S215, the absorbance-convergence output unit 220 acquires absorbance-convergence data K_{c} and outputs the absorbance-convergence data K_{c} to the display unit 226.

Subsequently, in step S216, the data recorder 216 records contact pressure data Pᵣ acquired by the contact pressure acquisition unit 219.

Subsequently, in step S217, the contact pressure-convergence output unit 222 acquires contact pressure-convergence data P_{c} and outputs the contact pressure-convergence data P_{c} to the display unit 226.

Subsequently, in step S218, the data recorder 216 records the contact image acquired by the image acquisition unit 218.

Subsequently, in step S219, the contact region output unit 224 outputs, to each of the differential region output unit 225 and the display unit 226, contact region data A acquired by calculation from the lip of the subject and the total reflection surface of the ATR prism 16, based on the contact image read from the data recorder 216.

Subsequently, in step S220, the differential region output unit 225 calculates differential region data A_{c} between the contact region data A and the predetermined target contact region and outputs the differential region data A_{c} to the display unit 226.

Subsequently, in step S221, the display unit 226 displays, on the display 506, each of light intensity data I, light intensity-convergence data I_{c}, absorbance data K, absorbance-convergence data K_{c}, contact pressure data Pᵣ, contact pressure-convergence data P_{c}, contact region data A, and differential region data A_{c}. Further, these data are output to the determining unit 227.

Subsequently, in step S222, the determining unit 227 determines whether all the conditions represented by Iₘᵢₙ≤I≤Iₘₐₓ, I≤I_{cth}, Kₘᵢₙ≤K≤Kₘₐₓ, K_{c}≤K_{cth}, Pₘᵢₙ≤P≤Pₘₐₓ, P_{c}≤P_{cth}, and Aₘᵢₙ≤A≤Aₘₐₓ are satisfied.

In step S222, when the determining unit 227 determines that all the above-described conditions are satisfied (Yes in step S222), the display unit 226 displays “light intensity OK”, “absorbance OK”, “contact pressure OK”, and “contact region OK” on the display 506 in step S223, and the process proceeds to step S224. By contrast, when the determining unit 227 determines that not all the above-described conditions are satisfied (No in step S222), the process returns to step S212 to execute steps subsequent to step S212 again.

Subsequently, in step S224, a storage device such as RAM 503 stores light intensity data for each of the first to third probe light at that time.

Subsequently, in step S225, the absorbance output unit 217a outputs, to the biological information output unit 221a, the absorbance data of the first to third probe light acquired by calculation based on the light intensity data of the first to third probe light stored in the RAM 503.

Subsequently, in step S226, the biological information output unit 221a acquires blood glucose level data based on the absorbance data of the first to third probe light and outputs the blood glucose level data to the display unit 226.

Subsequently, in step S227, the display unit 226 displays the blood glucose level data on the display 506.

Subsequently, in step S228, the display unit 226 ends displaying of time information to the end of the measurement that is input from the clock unit 228.

As described above, the processor 2a can perform the processing of measuring a blood glucose level.

The order of the processing from steps S212 to S219 may be changed in an appropriate manner, or the processing from steps S211 to S219 may be performed in parallel.

<Effect of Blood Glucose Level Measuring Device 100a>

As described above, according to the first embodiment, the blood glucose level is output based on the light intensity of the probe light P emitted from the ATR prism 16 that is in contact with the subject's lip. The display unit displays information relating to light intensity and absorbance. The contact pressure of the subject's lip with respect to ATR prism 16 is detected, and is displayed as contact pressure data Pr. A contact image of the subject's lip with respect to ATR prism 16 is captured, and is displayed as contact region data A of the subject's lip with respect to the total reflection surface of ATR prism 16. The contact region data A is generated based on the contact image.

The user of the blood glucose level measuring device 100a can adjust how to contact his lip with the ATR prism 16 while visually viewing information relating to the displayed light intensity, absorbance, contact pressure data Pᵣ, or contact region data A. Thus, the blood glucose level measuring device 100a can accurately measure a blood glucose level, with contact state fluctuation being reduced between the subject's lip and the ATR prism 16.

According to the first embodiment, at least one of light intensity-convergence data I_{c}, absorbance-convergence data K_{c}, or contact pressure-convergence data P_{c} is further displayed. Thus, the data can be obtained with the light intensity data I, the absorbance data K, and the contact pressure data Pᵣ being stable, and the blood glucose level can be accurately measured.

According to the first embodiment, the blood glucose level measuring device 100a makes a determination to start acquiring a blood glucose level, based on the contact pressure data Pᵣ, the contact region data A, the light intensity-convergence data I_{c}, the absorbance-convergence data K_{c}, and the contact pressure-convergence data P_{c}. In addition to contact pressure data Pᵣ and contact region data A, the blood glucose level measuring device 100a makes a determination to start acquiring a blood glucose level, based further on light intensity-convergence data I_{c}, absorbance-convergence data K_{c}, and contact pressure-convergence data P_{c}. Thus, acquisition of a blood glucose level can automatically start upon stable contact between the subject's lip and the ATR prism 16.

According to the first embodiment, a differential region between a contact region data A and a predetermined target contact region is displayed. This allows the subject to visualize a deviation of the contact region data A from the ideal contact condition, making it easier for the subject to adjust the contact region.

### Other Embodiments

According to the first embodiment described above, the determining unit 227 makes a determination to start acquiring a blood glucose level when the contact pressure data Pᵣ is within a predetermined contact pressure range, the contact region data A is within a predetermined contact region range, the light intensity data I is within a predetermined light intensity range, the light intensity-convergence data I_{c} is a predetermined light intensity threshold I_{cth} or less, the absorbance data K is within a predetermined absorbance range, the absorbance-convergence data K_{c} is a predetermined absorbance threshold K_{cth} or less, and the contact pressure-convergence data P_{c} is a predetermined contact pressure threshold P_{cth} or less. However, the present invention is not limited to this example.

The determining unit 227 may make a determination to start acquiring a blood glucose level when the contact pressure data Pᵣ is within a predetermined contact pressure range and the contact region data A is within a predetermined contact region range. Alternatively, the determining unit 227 may make a determination to start acquiring a blood glucose level when the contact pressure data Pᵣ is within a predetermined contact pressure range, and the contact region data A is within a predetermined contact region range, and in addition, at least one of the following conditions a) to c) is satisfied:
a) the light intensity-convergence data I_{c} is a predetermined light intensity threshold I_{cth} or less,
b) the absorbance-convergence data K_{c} is a predetermined absorbance threshold K_{cth} or less, or
c) the contact pressure-convergence data P_{c} is a predetermined contact pressure threshold P_{cth} or less.

While the embodiments have been described above, the present invention is not limited to the above specifically disclosed embodiments, and various variations and alterations are possible without departing from the scope of the claims. In the above-described first embodiment, not all the processes of light intensity, absorbance, contact pressure, or contact region are often required, and at least one of these elements may be processed and displayed. In this case, the unnecessary detector may be omitted from the configuration of the device.

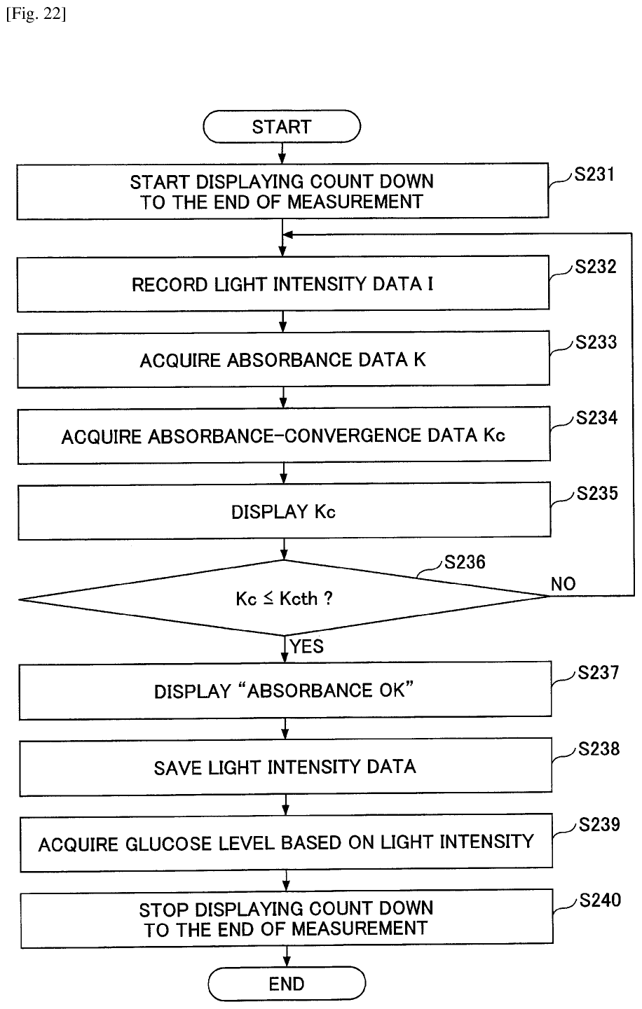

Herein, FIG. 22 is a flowchart illustrating an example of an operation of the blood glucose measuring device that displays absorbance only. Except for the operation of displaying absorbance only, other operations are the same as those illustrated in FIGS. 21A and 21B. Thus, duplicated descriptions are omitted.

In the above embodiments, functions such as a biological information acquisition unit 21 and a drive controller 23 are implemented, but the present invention is not limited to these examples. These functions may be implemented by separate processors, or the function of the biological information acquisition unit 21 may be dispersed in a plurality of processors. These functions may be implemented by a separate processor, or the functions of the biometric information acquisition unit 21 may be dispersed in a plurality of processing units. In addition, the function of the processor and the function of the storage device such as the data recorder 216 can be configured to be implemented by an external device such as a cloud server.

Further, the above embodiments illustrate examples of measuring blood glucose levels as biological information. However, the present invention is not limited to those examples. The embodiments can employ measurements of other biological information insofar as measurements are performed according to the ATR method.

In addition, an optical element such as a beam splitter configured to split a portion of the probe light emitted from the light source or from a hollow optical fiber, and a detecting element configured to detect the portion of the probe light are disposed such that probe light intensity of the split portion controls feedback of a drive voltage or a drive current of the light source, to reduce fluctuation in the probe light intensity. This reduces power fluctuation in the light source, thereby enabling more accurate biological information measurements.

The embodiments can also be applied to a blood glucose level measuring device that includes one light source to emit one wavelength of probe light from the one light source to measure a blood glucose level.

The embodiments also include a biological information measuring method. For example, the biological information measuring method includes emitting probe light, causing the incident probe light to be totally reflected by a total reflection member while the total reflection member is in contact with a subject, detecting light intensity of the probe light reflected from the total reflection member, outputting biological information acquired based on the light intensity, displaying a pressure from the subject with respect to the total reflection member, and a contact region between a total reflection surface of the total reflection member and the subject generated based on a contact image between the total reflection member and the subject. By such a biological information measuring method, the same effect as the biological information measuring device according to the first embodiment can be obtained.

The functions of the embodiments described above may also be implemented by one or more processing circuits. As used herein, a “processing circuit” includes a processor programmed to perform each function by software, such as a processor implemented in electronic circuits, an ASIC (Application Specific Integrated Circuit) designed to perform each function as described above, a digital signal processor (DSP), a field programmable gate array (FPGA), or a conventional circuit module.

### REFERENCE SIGNS LIST

1, la measuring unit
100, 100a blood glucose level measuring device (an example of biological information measuring device)
110 infrared light source (an example of a light source)
16 ATR prism
161 incident surface
162 first total reflection surface
163 second total reflection surface
164 emission surface
17 photodetector (example of light intensity detector)
18 camera (an example of the imaging unit)
2, 2a processor
21, 21a biological information acquisition unit
211 light source drive unit
212 light source controller
213 shutter drive unit
214 shutter controller
215 light intensity acquisition unit
216 data recording unit
217, 217a absorbance output unit
218 image acquisition unit
219 contact pressure acquisition unit
220 absorbance-convergence output unit
221, 221a biological information output unit
222 contact pressure-convergence output unit
223 light intensity-convergence output unit
224 contact region output unit
225 differential region output unit
226 display unit
2261 light intensity graph
2262 light intensity information
2263 light intensity-convergence information
2264 absorbance graph
2265 absorbance information
2266 absorbance-convergence information
2267 remaining time information
2268 contact pressure graph
2269 contact pressure information
2270 contact pressure-convergence information
2271 contact region map
2272 contact region information
2273 non-contact region information
2274 target contact region information
2275 differential region information
227 determining unit
228 clock unit
30 pressure sensor (example of pressure detector)
501 CPU
506 display
S living body (an example of a subject to be measured)
P probe light
I light intensity data
I_{c} light intensity convergence data
I_{cth} light intensity threshold
Pᵣ contact pressure data
P_{c} contact pressure-convergence data
P_{cth} contact pressure threshold
K absorbance data
K_{c} absorbance-convergence data
K_{cth} absorbance threshold
A contact region data
A_{c} differential region data

The present application is based on and claims priority of Japanese Priority Application No. 2019-195631 filed on Oct. 28, 2019, Japanese Priority Application No. 2019-195632 filed on Oct. 28, 2019, Japanese Priority Application No. 2019-195634 filed on Oct. 28, 2019, Japanese Priority Application No. 2019-195635 filed on Oct. 28, 2019, Japanese Priority Application No. 2019-201352 filed on Nov. 6, 2019, and Japanese Priority Application No. 2020-162222 filed on Sep. 28, 2020, the entire contents of which are hereby incorporated herein by reference.

1. A biological information measuring device comprising:
a light source configured to emit probe light;
a total reflection member configured to totally reflect the probe light with the total reflection member brought into contact with a subject to be measured;
a light intensity detector configured to detect light intensity of the probe light reflected from the total reflection member;
a biological information output unit configured to output biological information, the biological information being acquired based on the light intensity; and
a display unit configured to display the light intensity or an absorbance of the probe light, the absorbance being acquired based on the light intensity.

2. The biological information measuring device according to claim 1, further comprising:
a pressure detector configured to detect a pressure a pressure of the subject with respect to the total reflection member, wherein
the display unit further displays an output of the pressure detector.

3. The biological information measuring device according to claim 1, further comprising:
an imaging unit configured to capture an image of a vicinity of a contact region between a total reflection surface of the total reflection member and the subject to be measured, wherein
the display unit further displays information relating to the contact region, the contact region being generated based on an image captured by the imaging unit.

4. A biological information measuring device comprising:
a light source configured to emit probe light;
a total reflection member configured to totally reflect the probe light with the total reflection member brought into contact with a subject to be measured;
a light intensity detector configured to detect light intensity of the probe light reflected from the total reflection member;
a biological information output unit configured to output biological information, the biological information being acquired based on the light intensity; and
a display unit configured to display a pressure of the subject with respect to the total reflection member, and a contact region between a total reflection surface of the total reflection member and the subject, the contact region being generated based on a contact image between the total reflection member and the subject.

5. The biological information measuring device according to claim 4, further comprising:
a pressure detector configured to detect the pressure; and
an imaging unit configured to capture the contact image.

6. The biological information measuring device according to claim 4, further comprising:
an absorbance output unit configured to output an absorbance of the probe light, the absorbance being acquired based on the light intensity, wherein
the display unit further displays information relating to the light intensity and the absorbance.

7. The biological information measuring device according to claim 4, further comprising:
at least one of
a light intensity-convergence output unit configured to output light intensity-convergence, the light intensity-convergence being acquired based on the light intensity;
an absorbance-convergence output unit configured to output absorbance-convergence, the absorbance-convergence being acquired based on the light intensity; or
a pressure-convergence output unit configured to output pressure-convergence, the pressure-convergence being acquired based on the pressure, wherein
the display unit further displays at least one of the light intensity-convergence, the absorbance-convergence, or the pressure-convergence.

8. The biological information measuring device according to claim 4, wherein
the biological information output unit outputs the biological information acquired based on the light intensity detected in response to the pressure being within a predetermined pressure range, and the contact region being within a predetermined contact region.

9. The biological information measuring device according to claim 4, further comprising:
a determining unit configured to make a determination as to whether to start acquiring the biological information, the determination being made based on the pressure and the contact region.

10. The biological information measuring device according to claim 7, wherein
the biological information output unit outputs the biological information based on the light intensity acquired in response to the pressure being within a predetermined pressure range and the contact region being within a predetermined contact region, and at least one of following condition a), b), or c) being satisfied:
a) the light intensity-convergence is a predetermined light intensity threshold or less,
b) the absorbance-convergence is a predetermined absorbance threshold or less, and
c) the pressure-convergence is a predetermined pressure threshold or less.

11. The biological information measuring device according to claim 7, further comprising:
a determining unit configured to make a determination as to whether to start acquiring the biological information, the determination being made based on at least one of the light intensity-convergence, the absorbance-convergence, or the pressure-convergence, and based on a combination of the pressure and the contact region.

12. The biological information measuring device according to claim 4, further comprising:
a differential region output unit configured to output a differential region between the contact region and a predetermined target contact region, wherein
the display unit further displays the differential region.

13. The biological information measuring device according to claim 1, wherein
the biological information is blood glucose level information.

14. The biological information measuring device according to claim 13, wherein
a wavenumber of the probe light includes at least one of 1050 cm⁻¹, 1070 cm⁻¹, or 1100 cm⁻¹.

15. A method for measuring biological information, the method comprising:
emitting probe light;
totally reflecting the probe light by a total reflection member with the total reflection member brought into contact with a subject to be measured;
detecting light intensity of the probe light reflected from the total reflection member;
outputting biological information acquired based on the light intensity; and
displaying a pressure of the subject with respect to the total reflection member, and a contact region between a total reflection surface of the total reflection member and the subject, the contact region being generated based on a contact image between the total reflection member and the subject.
